(19) United States
(12) Patent Application Publication
(10) Pub. No.: US 2025/0166836 A1
(43) Pub. Date: May 22, 2025

(54) SYSTEMS AND METHODS FOR GENERATING PET LIKELIHOOD SCORES FOR DISEASES, CLINICAL CONDITIONS AND TRAITS

(71) Applicant: Mars, Incorporated, McLean, VA (US)

(72) Inventors: Rebecca Chodroff FORAN, Camas, WA (US); Jamie FREYER, Battleground, WA (US); Michelle DAYA, Aurora, CO (US); Jason Troy HUFF, Portland, OR (US); Julia LABADIE, Golden, CO (US)

(21) Appl. No.: 18/948,764

(22) Filed: Nov. 15, 2024

Related U.S. Application Data
(60) Provisional application No. 63/697,117, filed on Sep. 20, 2024.
(60) Provisional application No. 63/599,617, filed on Nov. 16, 2023.

(51) Int. Cl. G16H 50/30; G16B 20/20; G16B 40/20; G16H 10/20; G16H 10/60; G16H 15/00; G16H 20/00; G16H 50/70

(52) U.S. Cl. CPC G16H 50/30; G16B 20/20; G16B 40/20; G16H 10/20; G16H 10/60; G16H 15/00; G16H 20/00; G16H 50/70

(57) ABSTRACT

Systems and methods for generating a likelihood score that is indicative of a likelihood of a pet developing at least one of a disease, a clinical condition or other trait are disclosed. An example method may include: receiving, at a server system, pet data associated with the pet, wherein the pet data includes genetic data and breed data; generating, using a processor of the server system, a likelihood score for the pet associated with the clinical condition by applying the pet data to a trained machine learning model, wherein the trained machine learning model is trained to predict likelihood scores for the clinical condition; and causing, by the processor of the server system, a visual representation of the likelihood score to be displayed on a user device, wherein the visual representation includes an indication of the likelihood of the pet developing the clinical condition. Other aspects are described and claimed.

### CROSS-REFERENCE TO RELATED APPLICATION(S)

This patent application claims the benefit of priority to U.S. Provisional Patent Application No. 63/697,117, filed on Sep. 20, 2024, and U.S. Provisional Patent Application No. 63/599,617, filed on Nov. 16, 2023, the entireties of which are incorporated herein by reference.

### TECHNICAL FIELD

The present disclosure relates generally to the field of monitoring and managing pet health and, more specifically, to systems and methods for predicting a pet's likelihood of developing a disease, clinical condition or other trait using one or more trained machine learning models.

### BACKGROUND

Pets play a significant role in the lives of many individuals and families, providing, for example, companionship and emotional support. Just like humans, pets may be susceptible to various types of diseases or clinical conditions, including genetic disorders, chronic diseases, age-related ailments, and the like. Early detection and proactive management of these diseases or conditions are crucial for not only ensuring the well-being and longevity of pets, but also for providing timely intervention and effective treatment.

The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are disclosed for predicting the likelihood of a pet developing a particular disease, clinical condition or other trait. Specifically, the systems and methods may leverage machine learning and data analytics to predict the likelihood.

In summary, one aspect provides a computer-implemented method for identifying a likelihood of a pet developing a disease, clinical condition or other trait. The computer-implemented method includes: receiving, at a server system, pet data associated with the pet, wherein the pet data includes genetic data and breed data; generating, using a processor of the server system, a likelihood score for the pet associated with the disease, clinical condition or other trait by applying the pet data to a trained machine learning model, wherein the trained machine learning model is trained to predict likelihood scores for the disease, clinical condition or other trait; and causing, by the processor of the server system, a visual representation of the likelihood score to be displayed on a user device, wherein the visual representation includes an indication of the likelihood of the pet developing the disease, clinical condition or other trait.

In another aspect, a computer system for identifying a likelihood of a pet developing a disease, clinical condition or other trait is disclosed. The computer system includes: at least one processor; and at least one memory storing instructions that are executable by the at least one processor, cause the at least one processor to: receive pet data associated with the pet, wherein the pet data includes genetic data and breed data; generate, by applying the pet data to a trained machine learning model, a likelihood score for the pet associated with the disease, clinical condition or other trait, wherein the trained machine learning model is trained to predict likelihood scores for the disease, clinical condition or other trait; and cause a visual representation of the likelihood score to be displayed on a user device, wherein the visual representation includes an indication of the likelihood of the pet developing the disease, clinical condition or other trait.

In yet another aspect, a non-transitory computer-readable medium for identifying a likelihood of a pet developing a disease, clinical condition or other trait is disclosed. The non-transitory computer-readable medium stores computer-executable instructions which, when executed by at least one processor, cause the at least one processor to perform operations comprising: receiving pet data associated with the pet, wherein the pet data includes genetic data and breed data; generating a likelihood score for the pet associated with a disease, clinical condition or other trait by applying the pet data to a trained machine learning model, wherein the trained machine learning model is trained to predict likelihood scores for the disease, clinical condition or other trait; and causing a visual representation of the likelihood score to be displayed on a user device, wherein the visual representation includes an indication of a likelihood of the pet developing the disease, clinical condition or other trait.

Additional objects and advantages of the disclosed aspects will be set forth in part in the description that follows, and in part will be apparent from the description, or may be learned by practice of the disclosed aspects. The objects and advantages of the disclosed aspects will be realized and attained by means of the elements and combinations particularly pointed out in the appended claims.

It is to be understood that both the foregoing general description and the following detailed description are exemplary and explanatory only and are not restrictive of the disclosed aspects, as claimed.

### BRIEF DESCRIPTION OF THE DRAWINGS

The accompanying drawings, which are incorporated in and constitute a part of this specification, illustrate several aspects and together with the description, serve to explain the principles of the disclosure.

FIG. 1 depicts an exemplary environment for predicting disease, clinical condition or other trait likelihoods of pets, according to one or more aspects of the present disclosure.

FIG. 2 depicts a graphical overview of genome-wide association studies (GWAS) that were conducted, according to one or more aspects of the present disclosure.

FIG. 3 depicts results of a GWAS directed to a disease, clinical condition or other traits, according to one or more aspects of the present disclosure.

FIG. 4 depicts results of a GWAS directed to another disease, clinical condition or other trait, according to one or more aspects of the present disclosure.

FIG. 5 depicts results of a GWAS directed to another disease, clinical condition or other trait, according to one or more aspects of the present disclosure.

FIG. 6 depicts results of a GWAS directed to another disease, clinical condition or other trait, according to one or more aspects of the present disclosure.

FIG. 7 depicts results of a GWAS directed to another disease, clinical condition or other trait, according to one or more aspects of the present disclosure.

FIG. 8 depicts a table overview that provides summary information of a plurality of disease, condition or other trait-specific machine learning models, according to one or more aspects of the present disclosure.

FIG. 9 depicts a view of a user interface of a pet health management application platform, according to one or more aspects of the present disclosure.

FIG. 10 depicts another view of the user interface of the pet health management application platform, according to one or more aspects of the present disclosure.

FIG. 11 depicts another view of the user interface of the pet health management application platform, according to one or more aspects of the present disclosure.

FIG. 12 depicts another view of the user interface of the pet health management application platform, according to one or more aspects of the present disclosure.

FIG. 13 depicts another view of the user interface of the pet health management application platform, according to one or more aspects of the present disclosure.

FIG. 14 depicts another view of the user interface of the pet health management application platform, according to one or more aspects of the present disclosure.

FIG. 15 depicts another view of the user interface of the pet health management application platform, according to one or more aspects of the present disclosure.

FIG. 16 depicts an exemplary flowchart of a method for training a machine learning model to generate a likelihood score for a disease, clinical condition or other trait, according to one or more aspects of the present disclosure.

FIG. 17 depicts an exemplary flowchart of a method for identifying a likelihood of a pet developing a disease, clinical condition or other trait, according to one or more aspects of the present disclosure.

FIG. 18 depicts an example computing system, according to one or more aspects of the present disclosure.

FIG. 19 depicts a view of the user interface of the pet clinical condition and trait application platform, according to one or more aspects of the present disclosure.

FIG. 20 depicts another view of the user interface of the pet clinical condition and trait application platform, according to one or more aspects of the present disclosure.

FIG. 21 depicts another view of the user interface of the pet clinical condition and trait application platform, according to one or more aspects of the present disclosure.

FIG. 22 depicts another view of the user interface of the pet clinical condition and trait application platform, according to one or more aspects of the present disclosure.

FIG. 23 depicts another view of the user interface of the pet health management application platform, according to one or more aspects of the present disclosure.

FIG. 24 depicts another view of the user interface of the pet health management application platform, according to one or more aspects of the present disclosure.

FIG. 25 depicts another view of the user interface of the pet health management application platform, according to one or more aspects of the present disclosure.

FIG. 26 depicts another graphical overview of genome-wide association studies (GWAS) that were conducted, according to one or more aspects of the present disclosure.

FIG. 27 depicts another table overview that provides summary information of a plurality of disease and clinical condition machine learning models, according to one or more aspects of the present disclosure.

FIG. 28 depicts another table overview that provides summary information of a plurality of clinical condition and other trait-machine learning models, according to one or more aspects of the present disclosure.

### DETAILED DESCRIPTION

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as, “substantially” and “generally,” are used to indicate a possible variation of ±10% of a stated or understood value.

As used herein, the term “user” generally encompasses any person or entity, such as a pet owner and/or a pet care provider (e.g., a veterinarian), that may desire information, resolution of an issue, or engage in any other type of interaction with a provider of the systems and methods described herein (e.g., via an application interface resident on their electronic device, etc.). The term “pet” or the like generally encompasses a domestic animal, such as a domestic canine, feline, rabbit, ferret, horse, cow, or the like. The term “electronic application” or “application” may be used interchangeably with other terms like “program,” or the like, and generally encompasses software that is configured to interact with, modify, override, supplement, or operate in conjunction with other software.

As used herein, a “machine-learning model” generally encompasses instructions, data, and/or a model configured to receive input, and apply one or more of a weight, bias, classification, or analysis on the input to generate an output based on a procedure of one or more algorithms. The output may include, for example, an analysis based on the input, a prediction, suggestion, or recommendation associated with the input, a dynamic action performed by a system, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of machine-learning models may operate on an input linearly, in parallel (e.g., across an ensemble), via a network (e.g., a neural network), or via any suitable configuration. When the particular output desired is a classification prediction, we refer to “machine-learning predictive models,” which may include generating likelihood scores for disease, clinical conditions or other traits.

Pets are vulnerable to various diseases, clinical conditions and other traits that can significantly impact their health and well-being. Traditionally, pet health assessments have relied on manual evaluations by veterinarians or other similar pet care providers based on observed symptoms, medical history, and physical examinations. However, such evaluations may be limited by subjectivity, variability, and the inability to consider a large volume of data. Consequently, conventional evaluation techniques may lead to inconsistent results and/or delayed diagnoses. Additionally, some pet owners may be precluded from obtaining a professional evaluation from a veterinarian for their pet due to one or more of: local veterinarian availability, cost, and/or time constraints. Accordingly, there is a need for a more accessible, objective, and data-driven approach to assess and predict a pet's likelihood of developing one or more diseases, clinical conditions or other traits.

Recent advancements in machine learning and artificial intelligence (AI) have opened up new possibilities for leveraging various types of data (e.g., medical records, genetic information, breed information, environmental factors, etc.) to develop machine-learning predictive models for disease, clinical condition or other trait likelihoods. These models can analyze and identify patterns, correlations, and likelihood factors associated with specific diseases, clinical conditions or other traits, thereby enabling the prediction of a pet's likelihood of developing those diseases, conditions or other traits. Furthermore, the utilization of genetic information, particularly single nucleotide polymorphisms (SNPs), may add a new dimension to disease, clinical condition or other trait likelihood predictions. For example, SNPs are variations in a single DNA nucleotide base that can occur at specific positions in the genome. These genetic variants can contribute to an individual's susceptibility to certain diseases. By incorporating SNPs into the predictive models, the system can capture the genetic predisposition of a pet and provide more accurate likelihood assessments. These models may be further trained to leverage various additional information about pets to facilitate likelihood prediction, e.g., breed, sex, reproductive characteristics (e.g., whether spayed or neutered), location-based environmental factors, and the like.

Accordingly, the system of the present disclosure leverages the combination of machine-learning predictive models, including the integration of genetic information through SNPs, to provide a comprehensive solution for predicting a pet's likelihood of developing diseases, clinical conditions or other traits and for advising pet owners of steps they can take to proactively prevent or delay onset of these diseases, conditions or other traits and/or address or alleviate the symptoms associated with these disease, conditions or other traits once observed. More particularly, machine learning models employed by the system may be trained on diverse datasets that include information from a large number of pets with known diseases, clinical conditions or other traits. The models may be trained to recognize patterns and correlations between input factors (e.g., breed, age, sex, genetic markers, location-based environmental factors, medical history, etc.) and the development of specific diseases, clinical conditions or other traits. By leveraging these models, the system may generate a likelihood score indicating the likelihood of the pet developing a particular disease, clinical condition or other trait within a specified time frame. Indications of the generated likelihood score, and other associated information, may be provided to a user via a user-friendly application platform and interface. The user interface may allow veterinarians, pet owners, and/or other individuals with granted permissions to easily input and access pet data, view likelihood scores, and understand the implications of the predictions. Collectively, the system enables early identification, personalized risk assessment, and proactive management of pet health, thereby contributing to the improved well-being of a pet and potentially to the extension of their lifespan.

It is important to note that although the concepts described herein are directed to generating a likelihood score that is representative of the likelihood of a pet developing a disease, clinical condition or other trait, such a designation is not limiting. More particularly, the concepts described herein may also be utilized to predict a pet's likelihood of developing non-clinical conditions and/or other condition-related predictions.

The subject matter of the present disclosure will now be described more fully hereinafter with reference to the accompanying drawings, which form a part hereof, and which show, by way of illustration, specific exemplary aspects. An aspect or implementation described herein as “exemplary” is not to be construed as preferred or advantageous, for example, over other aspects or implementations; rather, it is intended to reflect or indicate that the aspect(s) is/are “example” aspect(s). Subject matter may be embodied in a variety of different forms and, therefore, covered or claimed subject matter is intended to be construed as not being limited to any exemplary aspects set forth herein; exemplary aspects are provided merely to be illustrative. Likewise, a reasonably broad scope for claimed or covered subject matter is intended. Among other things, for example, subject matter may be embodied as methods, devices, components, or systems. Accordingly, aspects may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one aspect” or “in some aspects” as used herein does not necessarily refer to the same aspect and the phrase “in another aspect” as used herein does not necessarily refer to a different aspect. It is intended, for example, that claimed subject matter include combinations of exemplary aspects in whole or in part.

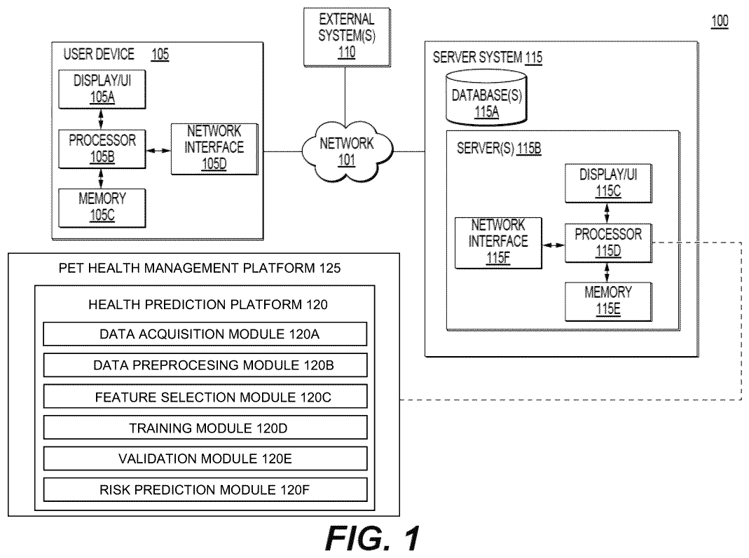

FIG. 1 depicts an exemplary environment 100 that may be utilized with the pet disease, clinical condition or other trait likelihood prediction techniques presented herein. A user device 105, one or more external system(s) 110, and a server system 115 may communicate across a network 101. As will be discussed in further detail below, the server system 115 may communicate with one or more of the other components of the environment 100 across the network 101. The user device 105 may be associated with a user, e.g., a system manager, a pet owner, a veterinarian, a researcher, or the like. Although depicted as separate components in FIG. 1, it should be understood that a component or portion of a component in the environment 100 may, in some aspects, be integrated with or incorporated into one or more other components. For example, a portion of the display 115C may be integrated into the user device 105 or the like. In some aspects, operations or aspects of one or more of the components listed above may be distributed amongst one or more other components. Any suitable arrangement and/or integration of the various systems and devices of the environment 100 may be used. Furthermore, it should be understood that while only one user device is shown in FIG. 1, the environment 100 may include a plurality of user devices 105 that are configured to communicate with the server system 115 over the network 101.

In some aspects, the components of the environment 100 may be associated with a common entity (e.g., a single business or organization, etc.). Alternatively, one or more of the components may be associated with a different entity than another. The systems and devices of the environment 100 may communicate in any arrangement. For example, the user device 105 may be associated with one or more clients or service subscribers, and the server system 115 may be associated with a service provider responsible for receiving and processing raw datasets from the one or more clients or service subscribers. As will be discussed herein, systems and/or devices of the environment 100 may communicate in order to collect and aggregate data from various sources (e.g., veterinary medical records, genetic database(s), environmental data sources, pet owner inputs, etc.), train one or more machine-learning models based on the aggregated data, and leverage the trained models to generate scores that predict a pet's likelihood of developing a disease, clinical condition or other trait, among other activities.

The user device 105 may be configured to enable the user to access and/or interact with other systems in the environment 100. For example, the user device 105 may be a computer system such as, for example, a desktop computer, a laptop, a mobile device, a tablet device, a wearable device, etc. The user device 105 may include a display/user interface (UI) 105A, a processor 105B, a memory 105C, and/or a network interface 105D. The user device 105 may execute, by the processor 105B, an operating system (O/S) and at least one electronic application (each stored in memory 105C). The electronic application may be a desktop program, a browser program, a web client, or a mobile application program (which may also be a browser program in a mobile O/S), system control software, system monitoring software, software development tools, or the like. In some aspects, the electronic application(s) may be associated with one or more of the other components in the environment 100, such as the server system 115. The application may manage the memory 105C, such as a database, to transmit streaming data to the network 101. The display/UI 105A may be a touch screen or a display with other input systems (e.g., mouse, keyboard, etc.) so that the user(s) may interact with the application and/or the O/S. The network interface 105D may be a TCP/IP network interface for, e.g., Ethernet or wireless communications with the network 101. The processor 105B, while executing the application, may generate data and/or receive user inputs from the display/UI 105A and/or receive/transmit messages to the server system 115, and may further perform one or more operations prior to providing an output to the network 101.

The electronic application, executed by the processor 105B of the user device 105, may generate one or many points of data that can be accessed, viewed, and/or interacted with by a user of the user device 105. More particularly, the electronic application may be associated with a pet health management platform 125 that is hosted, managed and/or supported by one or more of the server system 115. A user of user device 105 may interact with pet health management platform 125 via the electronic application to obtain various types of pet information (e.g., disease, clinical condition or other trait likelihood scores, etc.), provide pet health inputs, schedule veterinarian visits, and the like. In some aspects, such as illustrated in FIG. 1, the pet health management platform 125 may include an integrated health prediction platform 120 that may leverage one or more trained machine-learning models to generate likelihood scores representative of the likelihood of a pet developing a particular disease, clinical condition or other trait (e.g., disease, clinical condition or other trait likelihood scores), as further described herein. Alternatively, in other aspects, the pet health management platform 125 may be separate from the health prediction platform 120 and may communicate data to, and receive data from, the health prediction platform 120. In some aspects, the health prediction platform 120, the pet health management platform 125, or other platform of the server system 115, may be configured to generate one or more reports and/or notifications that contain the disease, clinical condition or other trait likelihood scores and/or other associated information (e.g., treatment recommendations, etc.). These reports and/or notifications may then be provided to the user device 105 via the application.

The external system(s) 110 may be, for example, one or more third party and/or auxiliary systems that integrate and/or communicate with the server system 115 in performing various information extraction tasks. The external system(s) 110 may be in communication with other device(s) or system(s) in the environment 100 over the network 101. For example, the external system(s) 110 may communicate with the server system 115 via API (application programming interface) access over the network 101, and also communicate with the user device 105 via web browser access over the network 101. Non-limiting examples of the external systems 110 may include one or more data repositories containing pet medical records, ancestry results for different breeds, breed-specific genetic information, data associated with pet activity, behavior, and/or other characteristics collected by sensors of smart devices worn by and/or interacted with by the pet, or the like.

In various aspects, the network 101 may be a wide area network (“WAN”), a local area network (“LAN”), a personal area network (“PAN”), or the like. In some aspects, the network 101 includes the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

In some aspects, the server system 115 includes and/or interacts with an application programming interface for exchanging data to other systems, e.g., one or more of the other components of the environment. The server system 115 may include and/or act as the host for an application platform (e.g., the pet health management platform 125, etc.) that may be accessible by the user device 105.

The server system 115 may include one or more database(s) 115A and one or more server(s) 115B. The server system 115 may be a computer, system of computers (e.g., rack server(s)), and/or or a cloud service computer system. The server system 115 may store or have access to database(s) 115A (e.g., hosted on a third party server or in memory 115E). The server(s) 115B may include a display/UI 115C, a processor 115D, a memory 115E, and/or a network interface 115F. The display/UI 115C may be a touch screen or a display with other input systems (e.g., mouse, keyboard, etc.) for an operator of the server(s) 115B to control the functions of the server(s) 115B. The server system 115 may execute, by the processor 115D, an operating system (O/S) and at least one instance of a servlet program (each stored in the memory 115E). When the user device 105 transmits input to the server system 115 (e.g., pet owner inputs, etc.), the received dataset and/or dataset information may be stored in the memory 115E or the database(s) 115A. The network interface 115F may be a TCP/IP network interface for, e.g., Ethernet or wireless communications with the network 101.

The processor 115D may include and/or execute instructions to implement a health prediction platform 120, which may include a data acquisition module 120A, a data preprocessing module 120B, a feature selection module 120C, a training module 120D, a validation module 120E, and a likelihood prediction module 120F. In an aspect, the data acquisition module 120A, the data preprocessing module 120B, the feature selection module 120C, the training module 120D, the validation module 120E, and the likelihood prediction module 120F may all be included within the server system 115, e.g., by the health prediction platform 120. Alternatively, some or all of the foregoing modules may be submodules of other modules within each other or may be resident on other components of the environment 100. For example, the data acquisition module 120A may be incorporated into an application resident on the user device 105 whereas the data preprocessing module 120B, the feature selection module 120C, the training module 120D, the validation module 120E, and the likelihood prediction module 120F may be contained within the health prediction platform 120.

The data acquisition module 120A may include instructions for collecting and aggregating a comprehensive set of information about pets from a diverse array of sources. For instance, data may be acquired from medical records obtained from veterinary clinics and/or hospitals, which may include information on previous diagnoses, treatments, medications, surgeries, and/or laboratory test results. Genetic information, particularly SNP data, may also be gathered through genotyping or DNA sequencing techniques. The SNP data may provide indications of specific genetic variations that can impact the predisposition of a pet to certain diseases, conditions or other traits. Genome-wide association studies (GWAS) rely on a large number of SNPs distributed throughout the genome to identify associations between genetic variants and the disease, clinical condition or other traits of interest. Environmental factors may also be gathered that are associated with pets located in a particular living situation and/or geographic region. For instance, data associated with a pet's living conditions, exposure to various types of toxins or pollutants, dietary information, exercise routines, and/or other relevant aspects that may influence a pet's health may be collected. Demographic information, including age, breed, sex, ancestry data, and/or other relevant characteristics may additionally be gathered to further enhance the understanding of potential likelihood factors associated with specific diseases, clinical conditions or other traits. The foregoing data types may be procured through collaborations with veterinary clinics, genetic databases, research institutions, and/or pet owners who voluntarily contribute their pet's data.

The data collected by the data acquisition module 120A may be passed to the data preprocessing module 120B to ensure compatibility and quality for machine-learning predictive models training. Stated differently, the data preprocessing module 120B may transform the raw collected pet data into a consistent and suitable format for training one or more machine learning models. The various steps involved in data preprocessing may include data cleaning (e.g., removal of any duplicate, incomplete, or erroneous entries from the dataset), missing value handling (e.g., resolving missing data points by employing appropriate techniques to estimate or fill in the missing values), normalization or standardization (e.g., rescaling the data to bring it to a common scale or distribution, which enables fair comparisons and prevents certain features from dominating the analysis due to their scales), and feature encoding (e.g., converting categorical variables or genetic variants, such as SNPs, into a numerical or binary representation that is suitable for machine learning models). It is important to note that the data preprocessing steps listed above may vary based upon the type of data collected by the data acquisition module 120A and/or by the type of machine learning model that will be trained on the preprocessed data. More particularly, the steps carried out by the data preprocessing module 120B may include more or less steps than what is listed and described above.

The preprocessed data may be passed to the feature selection module 120C to identify and select the most relevant features from the cumulative dataset for model training. This process reduces the dimensionality of the dataset by eliminating irrelevant or redundant features, which ultimately improves model performance, facilitates faster model training and inference (i.e., working with a reduced set of features reduces the computational complexity of training and inference processes), and contributes to enhanced model interpretation (i.e., users, such as veterinarians or pet owners, may be able to more easily understand and/or explain the factors contributing to a pet's likelihood of developing a disease, clinical condition or other trait). Several approaches may be leveraged to perform feature selection. For instance, one or more filter methods may be employed to assess the relevance of features based on statistical measures or correlations with the target variable(s) (e.g., common filter methods chi-square test, mutual information, correlation coefficients, analysis of variance (ANOVA), etc.). Additionally or alternatively, one or more wrapper methods may be utilized to evaluate the performance of machine learning models with different subsets of features to identify the optimal subset. Additionally or alternatively, one or more dimensionality reduction methods, such as principal component analysis (PCA) or singular value decomposition (SVD), may be used to transform the original features into a lower-dimensional space while preserving the most important information.

Further to the foregoing, in the context of SNP data, feature selection may involve identifying the most relevant and informative genetic variants that contribute to the prediction of a pet's likelihood of developing a disease, clinical condition or other trait. For instance, various measures may be employed to assess the importance of SNPs in predicting the disease, clinical condition or other trait of interest. One such measure may include the consideration of the p-value, obtained from one or more statistical tests (e.g., chi-square tests, Fisher's exact tests, etc.), which represents the association between each SNP and the disease, clinical condition or other trait. In general, SNPs with lower p-values may be considered more significant and may be selected as relevant features. In an aspect, one or more p-value thresholds (e.g., with threshold values ranging from 0.001, 0.01, 0.05, 0.1, 0.2, 0.3, 0.4, and 0.5) may be employed to further refine the specific SNPs to be utilized for model training. The value of each threshold may be based, for example, on the disease, condition or other trait analyzed for, with some diseases, conditions or other traits requiring a much lower p-value for certain SNPs to be considered than others. Additionally or alternatively to the foregoing, one or more machine learning algorithms, such as random forests or gradient boosting, may provide importance scores or feature rankings for each SNP. These scores reflect the contribution of each SNP to the predictive performance of the model, where SNPs having higher importance scores may be deemed to be more informative and can be selected for inclusion in the final feature set. Additionally or alternatively to the foregoing, linkage disequilibrium (LD) analysis may also be utilized to identify the correlation between SNPs within a genomic region. SNPs in strong LD with each other often capture similar information, and selecting only one representative SNP from a set of highly correlated SNPs can reduce redundancy.

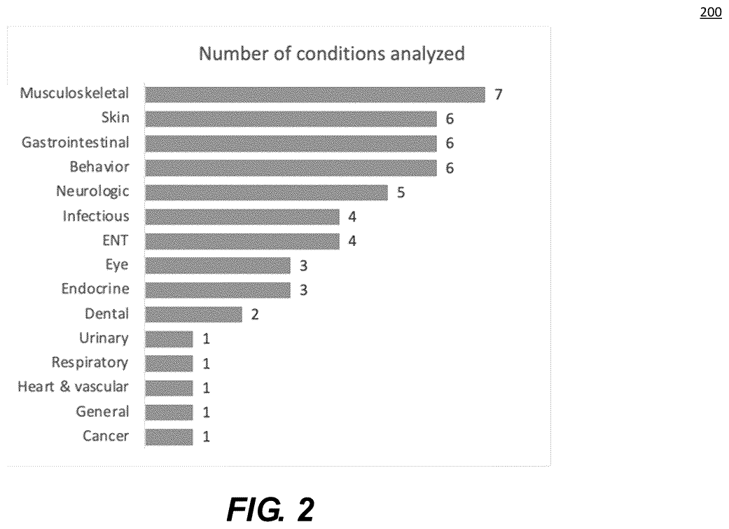

GWAS analysis may be conducted as part of the SNP feature selection process. These analyses involve the performance of statistical tests to assess the association between each SNP and the disease, clinical condition or other trait. The results of the GWAS analysis may provide insights into the SNPs that exhibit significant associations with the diseases, clinical conditions or other traits. FIGS. 2-7 illustrate graphical information associated with GWAS analyses conducted for a subset of various diseases, clinical conditions or other traits. Each of these GWAS analyses may be utilized to identify relevant SNPs for a given clinical condition that may subsequently be used to train a machine-learning predictive model for that disease, clinical condition or other trait.

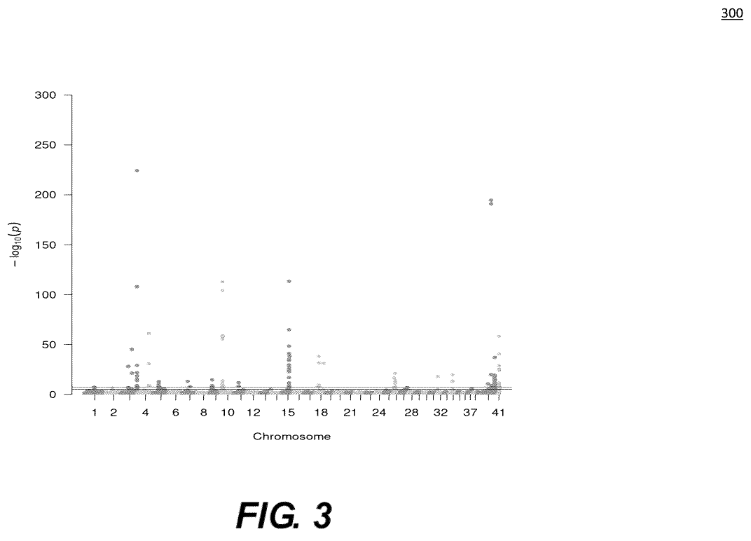

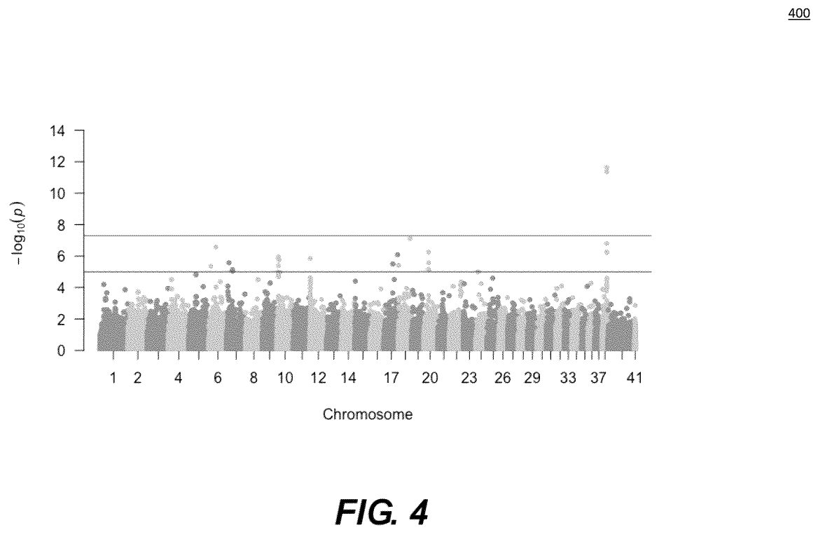

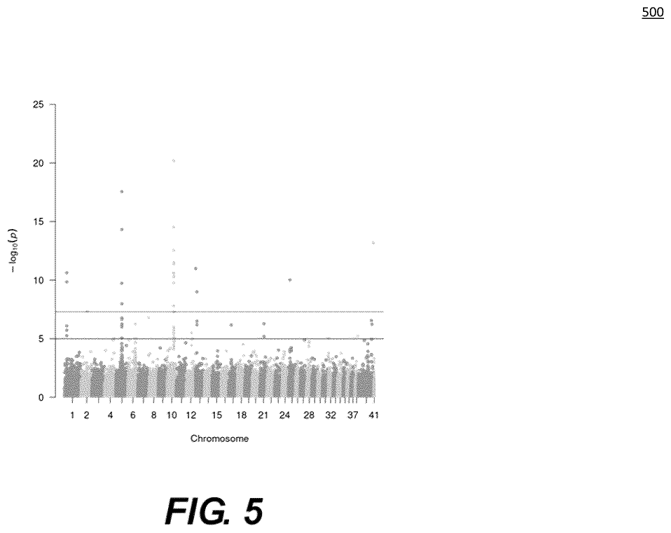

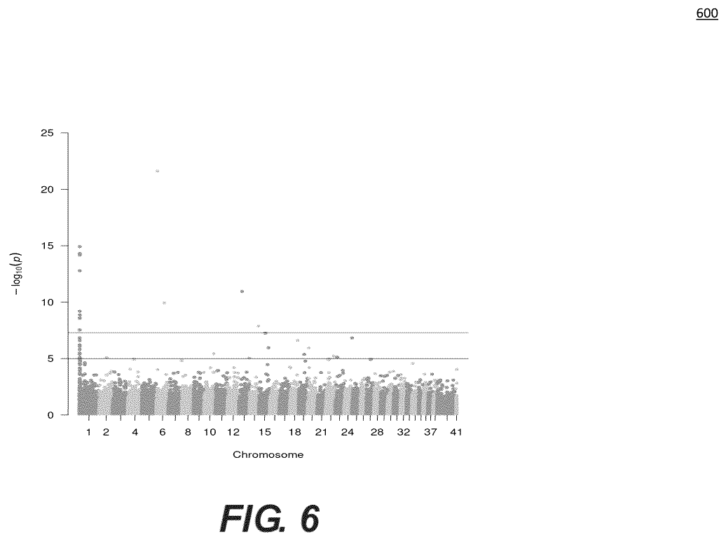

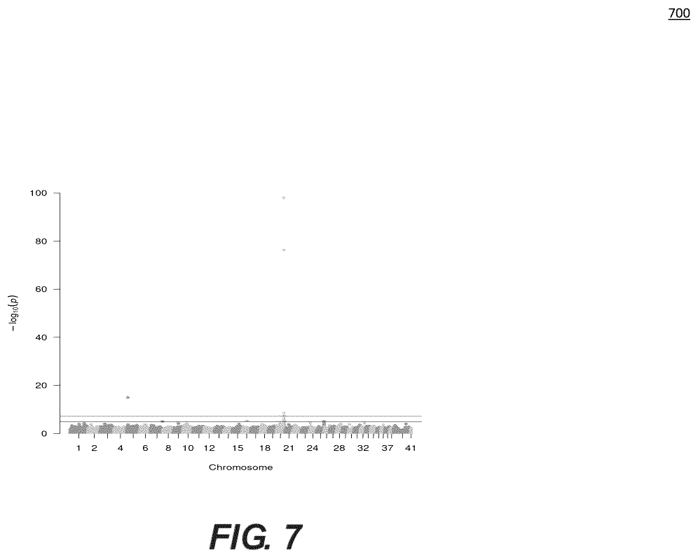

FIG. 2 provides a general overview of the GWAS analysis landscape via a graph 200 that includes an indication of an exemplary number of GWAS analyses conducted across various categories of pet diseases, clinical conditions or other traits. These categories are merely representative, and GWAS analyses may be performed for other types and/or categories of pet diseases, clinical conditions or other traits. Additionally, more or fewer GWAS analyses for diseases, clinical conditions or other traits may be performed to enable building of respective machine-learning predictive models for predicting likelihoods of those diseases, clinical conditions or other traits. FIG. 3 represents a data graph 300 of an atopic dermatitis GWAS. More particularly, a GWAS evaluating risk for atopic dermatitis among nearly 30,000 cases and an equal number of controls revealed a statistically significant peak on chromosome 38. The region of significance involved multiple genes in the signaling lymphocyte activation molecule family (SLAMF) of cell surface receptors. These genes are important in regulating immune functions and have been associated with autoimmune skin disorders in people. FIG. 4 represents a data graph 400 of a body weight GWAS. The GWAS here evaluated variants associated with body weight among dogs with a normal body condition score and replicated multiple known variants, including insulin like growth factor 1 receptor (IGF1R), ligand dependent nuclear receptor corepressor like (LCORL), growth hormone receptor (GHR), mothers against decapentaplegic homolog 2 (SMAD2), high-mobility group AT-hook 2 (HMGA2), fibroblast growth factor 4 (FGF4), and insulin-like growth factor 1 (IGF1). In addition, new variants were identified that had previously been associated with human height, including bone morphogenetic protein 3 (BMP3), testis expressed 2 (TEX2), endoplasmic reticulum to nucleus signaling 1 (ERN1), cholinergic receptor nicotinic beta 1 subunit (CHRNB1), and dynein axonemal heavy chain 2 (DNAH2). FIG. 5 represents a data graph 500 of an obesity GWAS. Multiple regions of interest were identified here. In particular, a melanocortin receptor gene on chromosome 1 and a leptin receptor gene on chromosome 5 that had previously been associated with human obesity were identified as regions of interest. FIG. 6 represents a data graph 600 of a motion sickness GWAS. In this study, a peak was identified on chromosome 1, which includes galanin receptor 1 (GALR1). GALR1 is widely expressed in the brain and has diverse functions including appetite and mood. The galanin hormone may play a role in response to orthostatic changes, including nausea. FIG. 7 represents a data graph 700 of a pancreatitis GWAS. In this study, two plausible peaks associated with pancreatitis were identified, one on chromosome 5 (e.g., on which the ST3GAL4 gene has been associated with hyperglycemia, inflammatory response, and is overexpressed in pancreatic adenocarcinoma) and on chromosome 20 (e.g., on which a cluster of genes was identified, including some involved in colonic dysfunction and protein metabolism).

The preprocessed dataset, including the data identifying the relevant SNPs from each GWAS analysis for a given disease, clinical condition or other trait along with other selected features, may be utilized as training data to train the machine-learning predictive models via the training module 120D. In general, the machine-learning predictive models may learn to recognize patterns and correlations between the encoded SNPs and the presence or absence of specific diseases, clinical conditions or other traits. In an aspect, the training module 120D may include one or more machine-learning predictive models and/or instructions associated with each of the one or more machine-learning predictive models, e.g., instructions for generating, training, and/or using the machine-learning predictive models. The server system 115 may include instructions for retrieving output features, e.g., based on the output of the machine-learning predictive models, and/or operating the display/UIs 105A and/or 115C to generate one or more output features, e.g., as adjusted based on the machine-learning predictive models. In some aspects, a system or device other than the server system 115 may be used to generate and/or train the machine-learning predictive models. For example, such a system may include instructions for generating the machine-learning predictive models, the training data and/or ground truth, and/or instructions for training the machine-learning predictive models. A resulting trained machine-learning predictive models may then be provided to the server system 115. In some examples, the trained machine-learning predictive models may be stored in the database(s) 115A and retrieved for subsequent execution by the likelihood prediction module 120F, for example.

In some aspects, the machine-learning predictive models may be based on architectures such as neural networks (e.g., convolutional neural network, etc.), support-vector machines (SVMs), decision trees, random forests, Gradient Boosting or Extreme Gradient Boosting (XGBoost), or any ensemble combination thereof. Using the machine-learning predictive models (e.g., XGBoost) may provide additional advantages. For example, the machine-learning predictive models may achieve a high accuracy by seamlessly combining one or more heterogeneous data sources without needing transformation or normalization of one or more input features. In addition, capturing nonlinear and interaction effects between the input features may effectively deal with input collinearity with less forms of regularization. Lastly, using breed ancestry data may provide a proxy for information that may not have otherwise been captured from microarray SNP data.

Alternate aspects include using techniques such as transfer learning, wherein one or more pre-trained machine learning models on large common or domain specific dataset may be leveraged for analyzing the training data. The training module 120D may be configured to cause the machine-learning predictive models to learn semantic associations between the raw data and the context with which it is associated with, such that the machine-learning predictive models are configured to provide output features that are contextually relevant for a user's purpose. In supervised learning, e.g., where a ground truth is known for the training data provided, training may proceed by feeding a sample of training data into a model with variables set at initialized values, e.g., at random, based on Gaussian noise, a pre-trained model, or the like. For instance, during the training phase, the selected machine learning model is presented with the labeled pet data from the training set. The model learns to capture the relationships between the input features (e.g., such as pet characteristics, genetic data, environmental factors, selected SNPs, etc.) and the corresponding target variable (e.g., the likelihood of developing a particular disease, clinical condition or other trait).

After the machine-learning predictive models are constructed and trained, the validation module 120E may assess the performance of each machine-learning predictive model. In an aspect, the machine-learning predictive models may be evaluated using appropriate performance metrics, considering both the SNP data and other input features. For instance, evaluation metrics such as the area under the receiver operating characteristic curve (AUC), sensitivity, specificity, and/or positive/negative predictive values may be used to assess the model's predictive capabilities. Furthermore, in an aspect, two different validation data sets may be employed in attempt to avoid overfitting, which occurs when a model performs well on the training data but fails to generalize to new data. The first validation data set may be a test set that was selected the same way as the training data set, but was held aside and not used for model training. The second validation data set may be a completely independent data set that was not used during training or model selection. This ensures that the model's performance is not biased by the data it has already seen. This independent data set may be, for example, an independent pet owner survey sample set. The machine-learning predictive models may be further optimized by fine-tuning various hyperparameters, including learning rate, regularization parameters, number of layers if using neural networks, number of trees or estimators if using random forests or gradient boosting techniques, and the like.

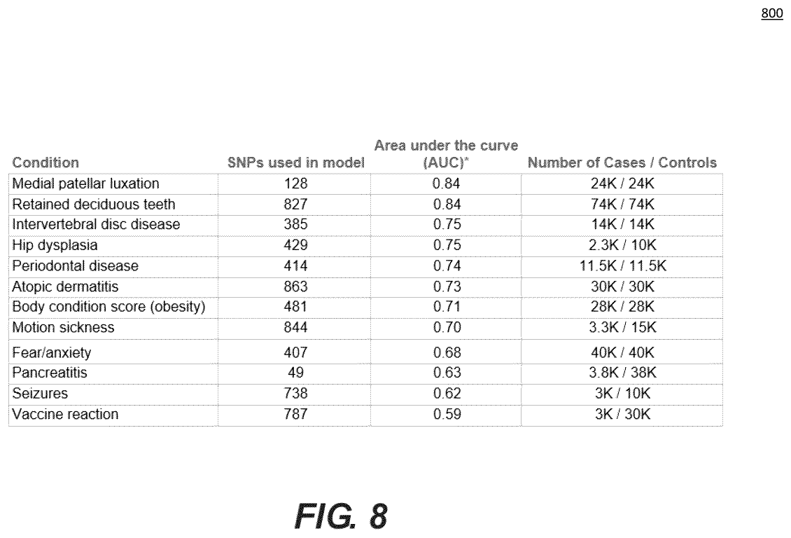

In an aspect, a single model (e.g., an optimal model) may be selected per disease, condition or other trait based on the highest AUC score achieved with the fewest number of SNP features (where fewer SNPs represent a more stringent GWAS p-value threshold). For instance, turning now to FIG. 8, an illustrative table 800 is presented that provides summary information for each optimal machine-learning predictive model chosen for 12 different diseases, clinical conditions or other traits. For each machine-learning predictive model, characteristic information such as the number of SNPs used in the model, the AUC score, and the number of cases and controls are provided. As briefly alluded to above, the AUC is a metric for classification accuracy of a binary predictive model across all score cutoffs. It measures a curve for all values of apparent true positive rates for equivalent false positive rates. It can vary from 0.5, indicating predictions are effectively random and the model has no predictive value, up to 1, indicating a perfectly predictive classifier. The AUC score provided for reach optimal machine-learning predictive model in the illustrative table 800 was calculated on a random subset of 10% of input samples held during model training for testing purposes.

Turning back to FIG. 1, the likelihood prediction module 120F may leverage the trained and validated machine-learning predictive models to predict a pet's likelihood of developing a disease, clinical condition or other trait. More particularly, a pet data set (e.g., cumulative data associated with a particular pet that is provided by the pet owner, a third party, and/or obtained from an online source) may be provided to the machine-learning predictive models, which may be configured to calculate a polygenic risk score (PRS) for each disease, condition or other trait. In an aspect, the PRS may be transformed into a binary indication of “at-risk” or “not at-risk” for the disease, condition or other trait the machine-learning predictive model is trained to monitor for. Additionally or alternatively, the risk likelihood may be represented on a scale from “significantly decreased likelihood” to “average likelihood” to “significantly increased likelihood.” Additionally or alternatively, the disease, clinical condition or other trait likelihoods may further be used as inputs downstream to recommend products (e.g., at home diagnostics, food, supplements, health and/or activity trackers, education content, etc.) to promote health, wellness, and precision care, as further described herein.

In an aspect, the results output by the likelihood prediction module 120F may be presented on Display/UI 105A and/or Display/UI 115C, e.g., via a user interface of the application associated with the pet health management platform 125. The user interface may provide an intuitive and accessible medium for veterinarians and pet owners to interact with the prediction results. The interface may be designed to be visually appealing, easy to navigate, and informative. In an aspect, the interface may allow users to create and maintain a comprehensive profile for each of their pets. This may include entering information such as breed, age, gender, weight, medical history, genetic markers, and environmental factors. In an aspect, the profile may be fully or partially constructed from a series of guided questions that are generated by the server system 115, and more specifically the health prediction platform 120, and output to the user (e.g., inquiring about observed symptoms in the pet, known environmental factors that have an effect on the pet, known disease, medical conditions or other traits experienced by the pet, past diagnoses, etc.). This profile may serve as the basis for generating accurate likelihood predictions. In an aspect, the user interface may also include a dashboard that serves as the central hub for accessing various functionalities and information. It may provide an overview of the pet's profile and displays the likelihood scores for different diseases, clinical conditions or other traits. Furthermore, the interface may be designed to be mobile-friendly, thereby allowing users to access the system from their smartphones, tablets, wearable devices, etc. This promotes convenience and flexibility in monitoring and managing a pet's health on the go.

Further to the foregoing, the user interface may display likelihood scores for different diseases, clinical conditions or other traits prominently. Each disease, condition or other trait may be accompanied by a visual representation, such as a color-coded indicator or a progress bar, to quickly convey the likelihood level so that users can easily interpret the results at a glance. Furthermore, the interface may incorporate interactive data visualization tools to present the correlations and trends between various input factors and the likelihood of developing diseases, clinical conditions or other traits. This allows users to explore the underlying data and gain insights into the predictive models. Additionally, the user interface may provide explanations and interpretations of the likelihood scores to help users better understand the implications. For instance, the user interface may include descriptions of the diseases, clinical conditions or other traits, the significance of the likelihood scores, and recommended actions based on the likelihood level. This information may empower users to make informed decisions regarding their pet's healthcare.

Further to the foregoing, the user interface may display a notification to alert users of any significant updates or changes in the pet's likelihood scores. This may ensure timely attention to potential health concerns and encourage proactive management of the pet's heath. For instance, the server system 115 and/or more specifically, the health prediction platform 120, may identify that a pet predicted to be at a high likelihood for developing a certain disease, condition or other trait may benefit from increased visits to the veterinarian. Therefore, a notification may be provided to the user to schedule a visit to the veterinarian, potentially ahead of a previously scheduled visit.

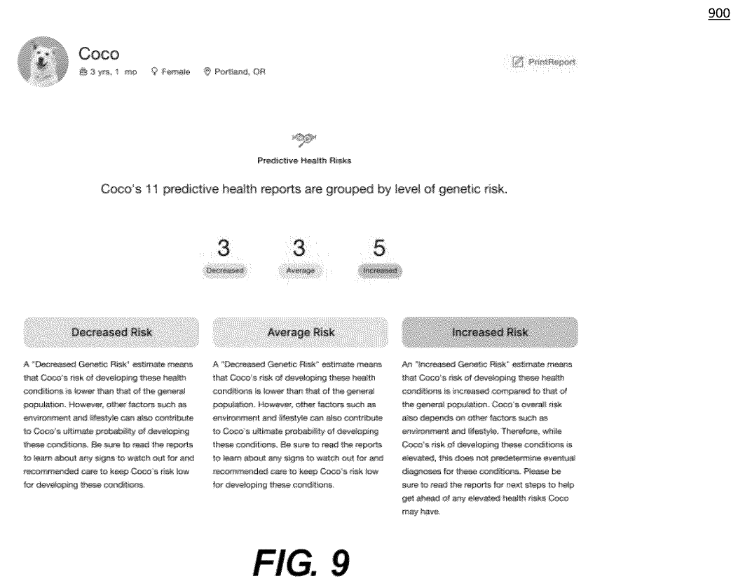

FIGS. 9-15 illustrate various user interfaces of the application associated with the pet health management platform 125. The application may be executing on the user device 105. In some examples, the user interfaces may be displaying various sections of a report and/or notifications generated by the health prediction platform 120, the pet health management platform 125, and/or or other platforms of the server system 115 based on predictions for one or more diseases, clinical conditions or other traits output by one or more of the machine-learning predictive models. It is important to note that the user interfaces illustrated in FIGS. 9-15 are non-limiting, and the icons, designs, and/or functionality depicted and/or described therein do not limit the use or presence of other icons, designs, and/or functionality of the application that are not explicitly described herein.

FIG. 9 depicts an overview page 900 indicating likelihood groupings for a plurality of diseases, clinical conditions or other traits. The overview page 900 may identify the number of diseases, clinical conditions or other traits that a particular pet's data was analyzed for and may categorize the results of these analyses into different groups (e.g., “increased likelihood”, “average likelihood”, “increased likelihood”, etc.) based on the predicted likelihood that the pet would develop each disease, clinical condition or other trait. For example, FIG. 9 illustrates that pet data associated with the user's pet was analyzed for 11 diseases, clinical conditions or other traits and the user's pet was identified as having: decreased likelihood for developing 3 clinical conditions, average likelihood for developing 3 clinical conditions, and increased likelihood for developing 5 clinical conditions. Each likelihood score may be generated by a machine-learning predictive model that has been trained to predict the likelihood that the particular pet would develop a corresponding disease, clinical condition or other trait. The overview page 900 may further comprise explanations of what each category means in terms of the pet's likelihood of developing the disease, clinical condition or other trait with respect to the general population, along with other factors that the user should be mindful of to prevent their pet's likelihood of developing a particular disease, condition or other trait from increasing.

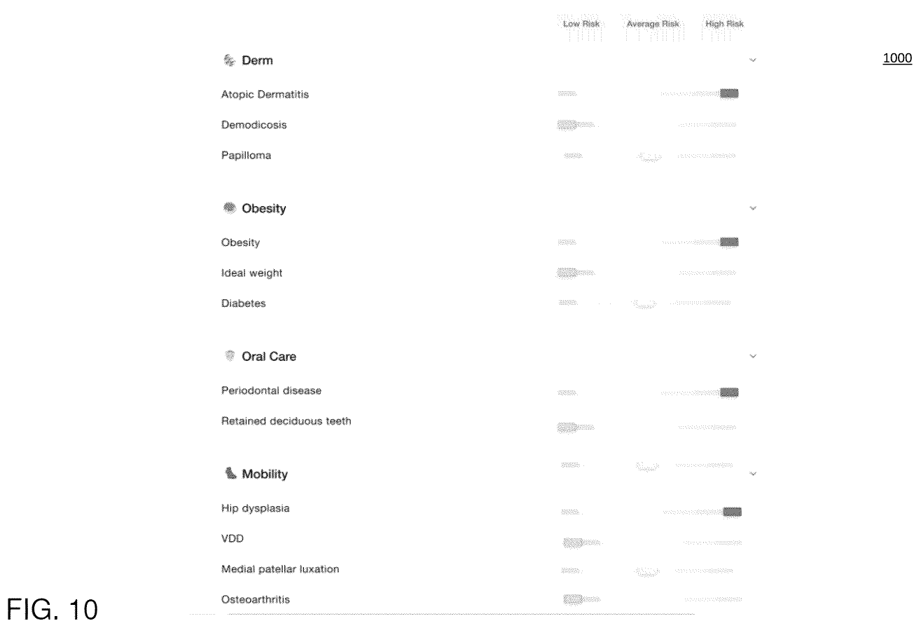

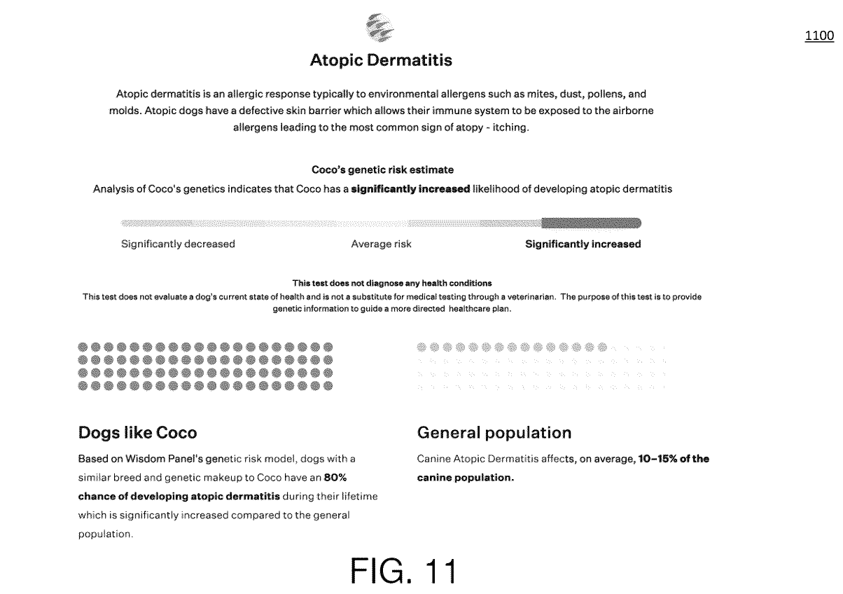

FIG. 10 depicts a likelihood indication screen 1000 that may include a listing of the diseases, conditions or other traits that were grouped into the likelihood designation categories, as illustrated in FIG. 9. More particularly, each disease, clinical condition or other trait presented on the likelihood indication screen 1000 may contain an indication of a likelihood designation for developing the disease, clinical condition or other trait that is represented by a progress bar, where a position of a slider or other similar component along the bar may indicate a level of likelihood from low to average to high. Additionally, the progress bar may utilize colors or other similar visual schemes to further visually emphasize the likelihood designation associated with each disease, condition or other trait (e.g., green for low likelihood, yellow for average likelihood, and red for high likelihood). The user interface may logically group the diseases, clinical conditions or other traits together (e.g., grouping clinical conditions related to derm/skin, obesity, oral care, mobility, etc.), which may enable a user to quickly obtain information about the likelihoods their pet has for developing a disease, clinical condition or other trait for each classification type. In an aspect, a user may obtain additional information associated with each disease, clinical condition or other trait by, for example, selecting the disease, clinical condition or other trait on the screen (e.g., via mouse input, touch input, stylus input, etc.). Upon selection, a clinical condition screen 1100 may be presented, as shown in FIG. 11. The clinical condition screen 1100 provides more specific information about the selected disease, clinical condition or other trait, the likelihood of the user's pet developing the selected disease, clinical condition or other trait, and how the likelihood for the user's pet compares to other similarly situated pets (e.g., dogs of the same breed, sex, age, etc.) as well as the general population of pets. For example, upon selection of the “atopic dermatitis” clinical condition in FIG. 10, the clinical condition screen 1100 may be presented in FIG. 11 that includes information related to atopic dermatitis, the pet's predicted likelihood of developing atopic dermatitis, and how the predicted likelihood for the user's pet compares to other similarly situated pets as well as the general population of pets.

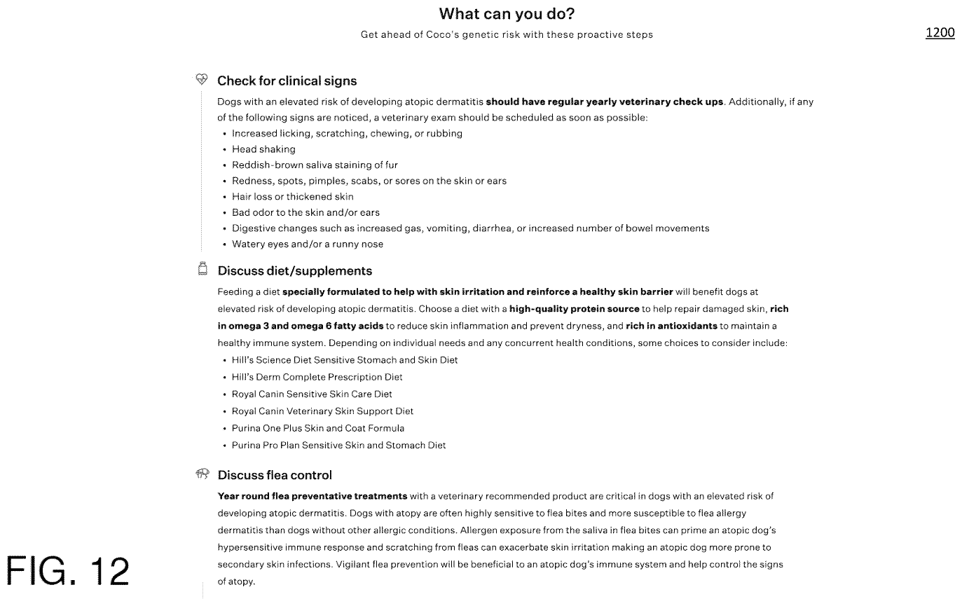

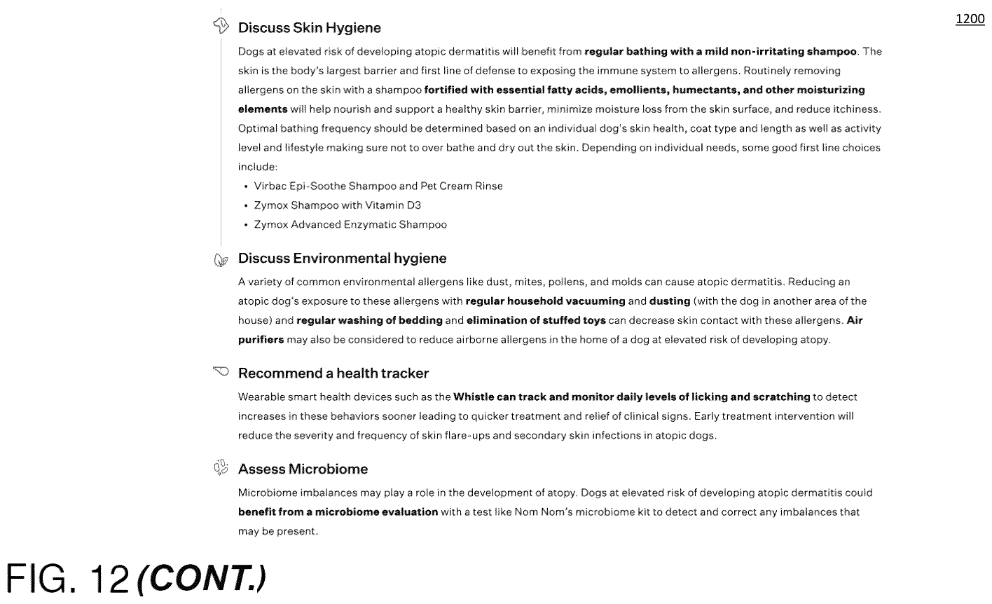

In an aspect, a user may also be presented with a recommendation screen 1200, as shown in FIG. 12, which may outline a variety of different steps the user can take to reduce the likelihood of their pet developing the disease, clinical condition or other trait and/or for alleviating symptoms that their pet may exhibit if they have the disease, clinical condition or other trait. For instance, the recommendation screen 1200 may provide guidance on clinical signs that the user should be on the lookout for, recommendations for diet alterations, drug and hygiene suggestions, assistive device suggestions, and microbiome assessment suggestions.

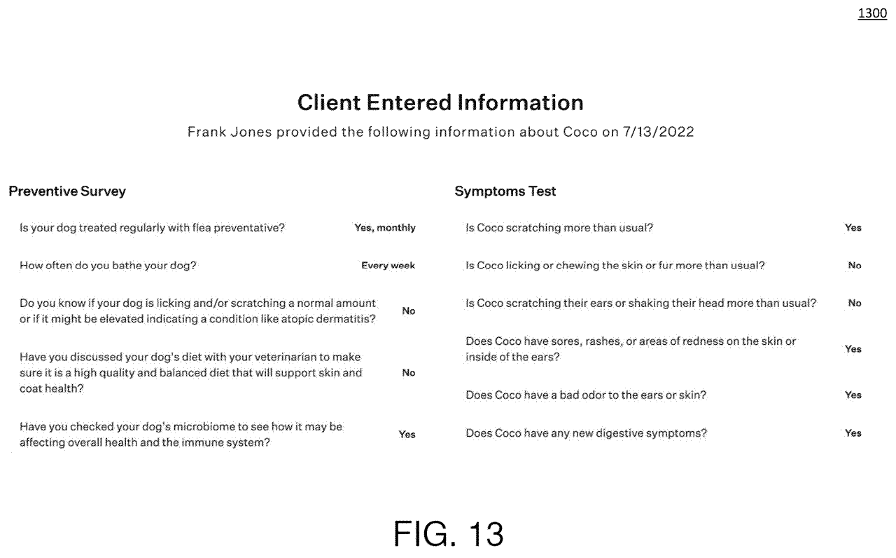

FIG. 13 provides an illustration of a client-provided information screen 1300 that may contain a summary of information provided by the user in response to one or more guided questions. For instance, in one aspect, the pet health management platform 125 may be configured to generate one or more surveys that may each contain several questions designed to elicit specific information from the user that may be utilized by the health prediction platform 120 to generate disease, clinical condition or other trait likelihood scores. These surveys may be, e.g., preventative surveys that include questions designed to obtain information regarding any preventative steps that are being taken to minimize the likelihood of development of a disease, clinical condition or other trait, symptom surveys that include questions designed to obtain information about specific symptoms a pet is experiencing, and/or other types of surveys designed to obtain other types of pet information. In an aspect, a user may be presented with these surveys once (e.g., upon registration of their pet with the pet health management platform 125), two or more times upon user request, two or more times at predetermined intervals (e.g., every 6 months, etc.), and/or two or more times in response to detected events (e.g., upon detection of a particular likelihood score assigned to the pet for a clinical condition, upon detection of a user's answer to a question in another survey, etc.).

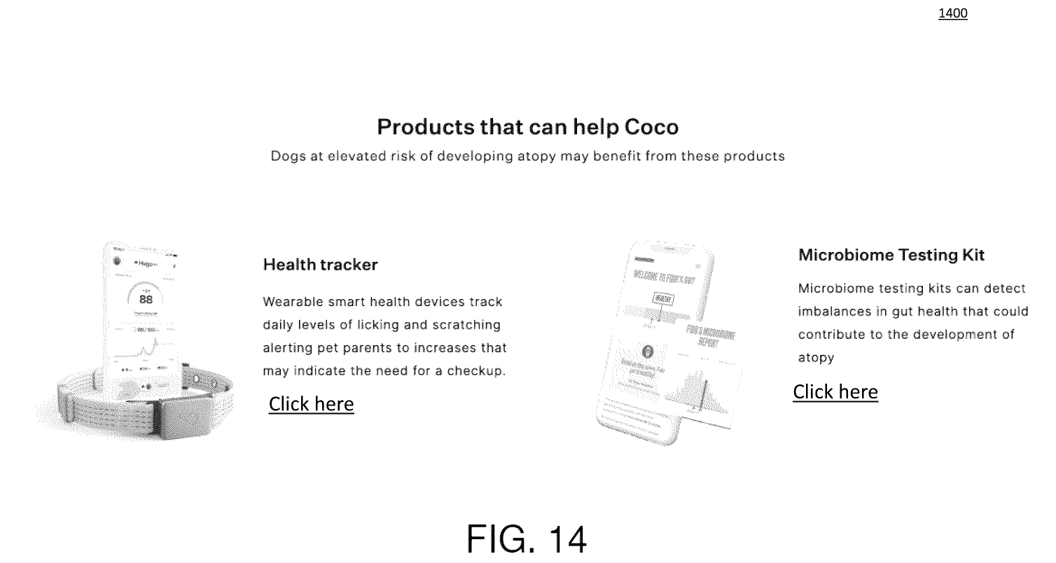

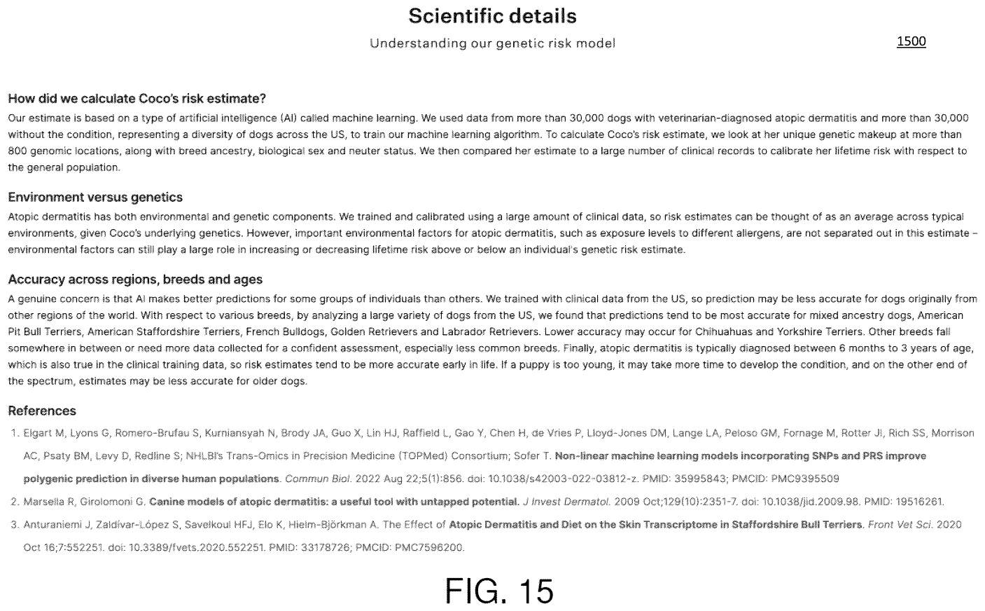

FIG. 14 provides an illustration of a product recommendation screen 1400 that may include images and/or information associated with products that the server system 115, pet health management platform 125, and/or health prediction platform 120 identifies as potentially beneficial to the pet (e.g., to promote proactive health monitoring and management) based on the pet's likelihood designation for a specific disease, clinical condition or other trait. The information associated with the products may also include links directing the user to a website of a merchant offering the products for sale, for example. Additionally or alternatively, the server system 115 and/or health prediction platform 120 may provide a scientific details screen 1500, as shown in FIG. 15, which provides an explanation of how the likelihood prediction was calculated for the user's pet, how other factors, such as environment, may increase or decrease the pet's genetic-based likelihood, and how the accuracy of that prediction may vary based on various pet characteristics (e.g., breed), along with references used to generate information provided in one or more sections of the health report (e.g., on one or more of the user interfaces shown in FIGS. 9-15).

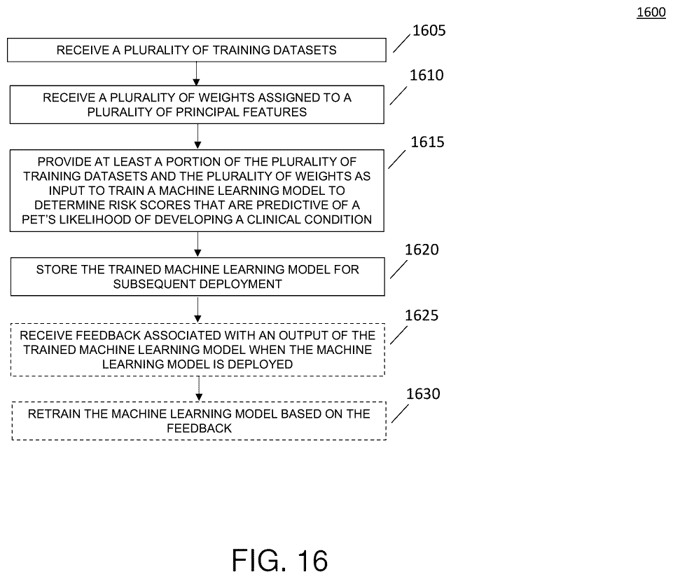

FIG. 16 depicts an exemplary process flow 1600 for training one or more machine-learning predictive models to generate a likelihood score that is representative of a pet developing a disease, clinical condition or other trait. In some examples, a separate machine learning model may be trained to predict each of a plurality of diseases, clinical conditions or other traits (e.g., each machine learning model is disease, clinical condition or other trait-specific), where each machine learning model may be trained using the exemplary process flow 1600. The exemplary process flow 1600 may be implemented by one or more components of the environment 100. The process flow 1600 is provided merely as an example, and can include additional, fewer, different, or differently arranged aspects than depicted in FIG. 6. In some embodiments, the training module 120D of the health prediction platform 120 may perform process flow 1600 to train the machine learning model. In other embodiments, a system or device other than the training module 120D performs the process flow 1600 to train the machine learning model. The trained machine learning model is then provided to the likelihood prediction module 120F for deployment. In some examples, the trained machine learning model may be monitored upon deployment and updated and/or retrained based on the monitoring to improve an accuracy of the trained machine learning models.

At step 1605, the process flow 1600 may include receiving a plurality of training datasets. The training datasets may include a variety of different types of pet data, including features such as genetic data (e.g., including SNP data obtained from a GWAS for one or more diseases, clinical conditions or other traits), clinical history data, demographic data, medical history data, environmental factor data, and/or lifestyle factor data. Each of the training datasets may be associated with a corresponding label that indicates the presence or absence of a disease, clinical condition or other trait of interest. In an aspect, the labeling process may be facilitated in a variety of different ways, e.g., by utilizing veterinary experts or based on diagnostic tests or observations retrieved from pet medical records. In some aspects, the labeled data may be represented in a structured format, such as a tabular dataset (e.g., where each row corresponds to an individual pet and the columns represent the various feature and the corresponding label. In an aspect, the labeled dataset may be divided into two subsets: a training set and a validation set. The training datasets may be used to train the one or more machine learning models to predict a pet's likelihood of developing the disease, clinical condition or other trait of interest, whereas the validation set may be held out to evaluate the trained models' performance. In an aspect, the data splitting may be conducted randomly, e.g., to ensure that the distribution of positive and negative cases is preserved in both datasets.

At step 1610, the process flow 1600 may include receiving a plurality of weights assigned to a plurality of principle features. Once the labeled data is prepared, feature weighting techniques may be applied to evaluate the importance or contribution of each feature in the prediction task. Specifically, these techniques aim to identify which features have the most predictive value (i.e., the features that have the most significant impact on predicting the pet's likelihood of developing the disease, clinical condition or other trait). Possible feature selection techniques that may be leveraged here include univariate feature selection, model-based feature selection, ensemble feature selection, and the like. Based on the importance scores, or rankings, obtained from the feature weighting techniques, a subset of the most informative features may be selected for model training, i.e., the “principal” features. This reduces the dimensionality of the feature space and focuses the model on the most relevant information. In an aspect, feature weighting may be an iterative process, where the initial feature set is evaluated, the models are trained, and the feature importance is reassessed. For instance, new data may indicate that a pet's environment is more predictive of the development of a particular clinical condition than previously known. In such an instance, the weight assigned to the environmental feature may be greater upon model retraining.

At step 1615, the process flow 1600 may include providing at least a portion of the plurality of training datasets and the plurality of weights as input to train the machine learning model to determine likelihood scores (e.g., polygenic likelihood scores) that may be reflective of a pet's likelihood of developing a particular disease, clinical condition or other trait (e.g., the clinical condition for which the training datasets having corresponding labels for). In some aspects, the machine learning model may be based on architectures such as neural networks (e.g., convolutional neural network, etc.), support-vector machines (SVMs), decision trees, random forests, or Gradient Boosting, Extreme Gradient Boosting (XGBoost), or any ensemble combination thereof. Alternate aspects include using techniques such as transfer learning, wherein one or more pre-trained machine learning models on large common or domain specific dataset may be leveraged for analyzing the training datasets. The training module 120D may be configured to cause the machine learning model to learn semantic associations between the raw data and the context with which it is associated with, such that the machine learning model is configured to provide output features that are contextually relevant for a user's purpose. In supervised learning, e.g., where a ground truth is known for the training datasets provided (e.g., via the corresponding labels), training may proceed by feeding a sample of training data (e.g., one of the training datasets) into a model with the features or variables set at initialized weights or values, e.g., at random, based on Gaussian noise, a pre-trained model, or the like. For instance, during the training phase, the selected machine learning model is presented with the labeled pet data from at least a portion of the training datasets. The model learns to capture the relationships between the input features (e.g., such as pet characteristics, genetic data, environmental factors, selected SNPs, etc.) and the corresponding target variable (e.g., the risk of developing a particular clinical condition).

In other examples, unsupervised, semi-supervised, and/or reinforcement learning processes can be employed to train the machine learning model. For unsupervised learning processes, the training datasets do not include pre-assigned labels or scores to aid the learning process. Rather, unsupervised learning processes include clustering, classification, or the like to identify naturally occurring patterns in the training datasets. Supervised or unsupervised K-means clustering or K-Nearest Neighbors can also be used. Combinations of K-Nearest Neighbors and an unsupervised cluster technique can also be used. For semi-supervised learning, a combination of the training datasets with pre-assigned labels or scores and similar datasets without pre-assigned labels or scores are used to train the machine learning model. When reinforcement learning is employed, an agent (e.g., an algorithm) is trained to make a decision regarding a score associated with a pet's likelihood of developing a disease, clinical condition or other trait from the training datasets through trial and error.

Once trained, at step 1620, the process flow 1600 may include storing the training machine learning model for subsequent deployment. For example, the trained machine learning model may be stored in one of database(s) 115A. In some examples, the trained machine learning model may be stored in association with the particular disease, clinical condition or other trait to enable easy retrieval of the machine learning model for deployment to predict a score associated with a pet's likelihood of developing that particular disease, clinical condition or other trait. In some examples, new training datasets and associated principal features may be received periodically (e.g., at a predetermined intervals, in response to predetermined events, etc.), and the trained machine learning model may be updated, modified, and/or retrained based on this new data. The trained machine learning model may be retrieved from the database(s) 115A and subsequently deployed (e.g., executed) by health prediction platform 120.

At optional step 1625, the process flow 1600 may include receiving feedback associated with an output of the trained machine learning model when the machine learning model is deployed. More particularly, a monitoring protocol may be engaged for the trained machine learning model, during which an actual outcome (e.g., whether or not the pet actually developed the particular disease, clinical condition or other trait) may be collected as feedback. Specifically, during a monitoring process, the likelihood score predicted for a given pet data set is analyzed along with the given pet data set and the actual outcome for the pet to determine an accuracy with which the trained machine learning model predicted the pet's likelihood of developing a particular disease, clinical condition or other trait. In other examples, other type of feedback, such as feedback from a veterinarian or other similar care provider, related to the pet's likelihood to develop the particular disease, condition or other trait may be collected and used for analysis of model accuracy.

At optional step 1630, the process flow 1600 may include re-training the trained machine learning model based on the feedback received at step 1610. In some examples, based on the analysis of the feedback performed during the monitoring process, the given pet data set and the actual outcome are provided as a new training dataset to retrain the trained machine learning model using the previously described machine learning model training process. For example, the weight or value of one or more features or variables of the trained machine learning model may be adjusted. In some examples, the trained machine learning model is retrained after a predefined number of new training datasets have been received. The retrained second machine learning model may then be stored for subsequent deployment (e.g., the process flow 1600 returns to step 1608).

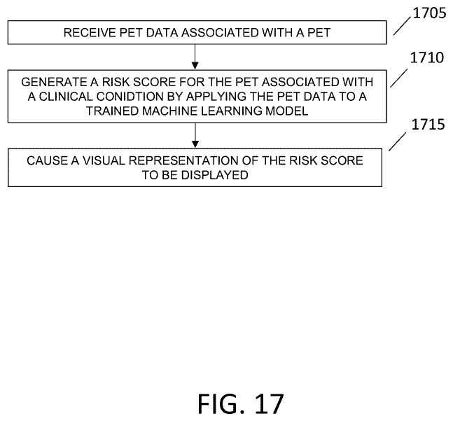

FIG. 17 depicts an exemplary process flow 1700 for generating a likelihood score associated with a disease, clinical condition or other trait. The exemplary process flow 1700 may be implemented by one or more components of the environment 100.

At step 1705, the server system 115 may receive pet data associated with a pet. The pet data may be pet-specific data and/or generalized data based on various pet characteristics. For instance, with respect to pet-specific data, the pet data may contain data that is specific to a particular pet. This kind of data may include observed symptoms, medical history, genetic information, dietary habits, lifestyle routines, ancestry results, and the like. Some or all of this specific pet data may be supplied by a user (e.g., a pet owner) via the application associated with the pet health management application platform. More particularly, the user may interact with icons and/or input fields of a user interface of the application to provide information about their pet. In some aspects, the application may be configured to provide a series of guided questions to the user, thereby helping to guide their input. For instance, the application may provide targeted inquiries that may help a user more effectively describe various symptoms that their pet is experiencing (e.g., “Is your dog scratching or shaking their head more than usual?”, “Does your dog have a bad odor to the ears or skin?”, “Does your dog have any new digestive symptoms?”, etc.). With respect to generalized data, the server system 115 may obtain more generalized information about the pet that corresponds to their inherent characteristics. For example, the server system 115 may obtain (e.g., from one or more available sources) general data associated with the pet's breed (e.g., expected symptoms, behavioral characteristics, or predisposed conditions based on the pet's breed), age (e.g., expected symptoms, behavioral characteristics, or predisposed conditions the pet may develop at their age), regional pet information (e.g., symptoms and/or characteristics exhibited by pets in a particular geographic region), and the like.

At step 1710, the server system 115 may generate a likelihood score for the pet associated with a disease, clinical condition or other trait by applying the pet data to a trained machine-learning predictive model. In an aspect, the trained machine-learning model may be trained to analyze the input data with respect to a particular disease, clinical condition or other trait in order to predict or generate the likelihood score for the pet associated with the disease, clinical condition or other trait. A non-limiting subset of diseases, clinical conditions or other traits that may be monitored for may include: medial patellar luxation, retrained deciduous teeth, intervertebral disc disease, hip dysplasia, periodontal disease, atopic dermatitis, body condition score (obesity), motion sickness, fear/anxiety, pancreatitis, seizures, and vaccine reaction. In an aspect, each of the trained machine learning models may be of a particular type, e.g., a gradient boosting model, a random forest model, a neural network, a logistic regression model, support vector machine, and a decision tree model. Each of the machine learning models may be trained using the techniques and processes previously described above. In particular, each model may be trained on genetic information comprising SNP data obtained from a GWAS for the particular disease, clinical condition or other trait. The specific SNP biomarkers utilized for training may provide indications of specific genetic variations that can impact the predisposition of a pet to certain diseases, conditions or other traits. In an aspect, the pet data may be analyzed by a specific model or subset of models (e.g., the model trained to predict likelihood scores associated with a disease, clinical condition or other trait of concern for a pet) or, alternatively, the pet data may be applied to and analyzed by all available models. For instance, a condition of concern may be identified based on user input received, such as answers to guided questions in a symptoms survey. For example, responsive to receiving an indication from a user that their pet's itching has increased, the server system 115 may retrieve (e.g., from the one of the databases 115A) a machine-learning predictive model trained for predicting atopic dermatitis, and/or a subset of models for predicting various dermatological conditions, to analyze the pet data.

Continuing with Step 1710, the server system 115 may utilize the trained machine learning model to generate a likelihood score. In an aspect, the risk score may be a numerical polygenic likelihood score that represents the likelihood that a pet will develop a specific disease, clinical condition or other trait. At step 1715, the server system 115 may cause a visual representation of the likelihood score to be displayed on the user device 105 (e.g., via the application). In an aspect, the likelihood information presented to the user may vary based on a context or role of the user. For instance, a pet owner may be presented with a more user-friendly interface that contains genericized and distilled information about the likelihood. For example, the pet owner may receive color-coded indications that represent various levels of likelihood (e.g., green corresponding to low likelihood, yellow corresponding to average likelihood, and red corresponding to high likelihood), straight-forward line and/or bar graphs that illustrate a pet's likelihood level, distilled explanations about the implications the risk score has for their pet, and the like. Conversely, an expert, such as a veterinarian or research scientist, may receive more objective information about the likelihood (e.g., the actual likelihood score, the raw data, etc.) in addition to, or in lieu of, the information presented to the pet owner.

In an aspect, the server system 115 may be configured to calculate a new likelihood score for a disease, clinical condition or other trait any time new pet data is received. More particularly, as new pet data is received (e.g., from the user in response to additional guided questions, from new developments in medical records, from new genetic testing data, etc.), a new likelihood score may be calculated for the pet. The automatic calculation of the new likelihood in response to receipt of any new pet data may ensure that the likelihood score is substantially always reflective of the pet's current likelihood of developing a disease, clinical condition or other trait. Alternatively, in another aspect, the server system 115 may be configured to calculate a new likelihood score only when specific types of new data are received. For example, the server system 115 may collect and store new responses to survey questions but may only initiate the calculation of a new likelihood score when new genetic data for the pet is received. Additionally or alternatively, the server system 115 may be configured to calculate a new likelihood score at a predetermined interval, irrespective of the volume and/or type of new data that is received in the interim.

Additionally to the foregoing, the server system 115 may be configured to generate and provide, via the application, other types of informative output to the user, based on the likelihood score information, in an effort to decrease the likelihood of a pet developing a disease, clinical condition or other trait. For instance, the user may be presented with various recommendations for: actions they can take in response to certain observed symptoms, dietary recommendations for their pet, hygiene recommendations for their pet, suggestions and purchase information for health-based products they can acquire to more closely monitor their pet's health, suggestions for other tests that may be run to obtain more data about their pet's health, and the like.

In general, any process discussed in this disclosure that is understood to be computer-implementable, such as the process illustrated in FIG. 15, may be performed by one or more processors of a computer system, such as one or more components of the environment 100, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such one or more components of the environment 100, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

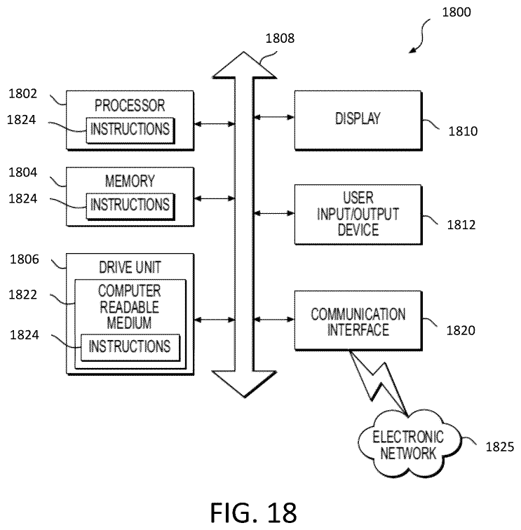

FIG. 18 is a simplified functional block diagram of a computer system 1800 that may be configured as a computing device for executing any processes or operations described herein, including the process illustrated in FIGS. 16-17, according to exemplary aspects of the present disclosure. In various aspects, any of the systems herein may be an assembly of hardware including, for example, a data communication interface 1820 for packet data communication. The platform also may include a central processing unit (“CPU”) 1802, in the form of one or more processors, for executing program instructions. The platform may include an internal communication bus 1808, and a storage unit 1806 (such as ROM, HDD, SDD, etc.) that may store data on a computer readable medium 1822, although the computer system 1800 may receive programming and data via network communications via electronic network 1825 (e.g., voice, video, audio, images, or any other data over the electronic network 1825). The computer system 1800 may also have a memory 1804 (such as RAM) storing instructions 1824 for executing techniques presented herein, although the instructions 1824 may be stored temporarily or permanently within other modules of computer system 1800 (e.g., processor 1802 and/or computer readable medium 1822). The computer system 1800 also may include input and output ports 1812 and/or a display 1810 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

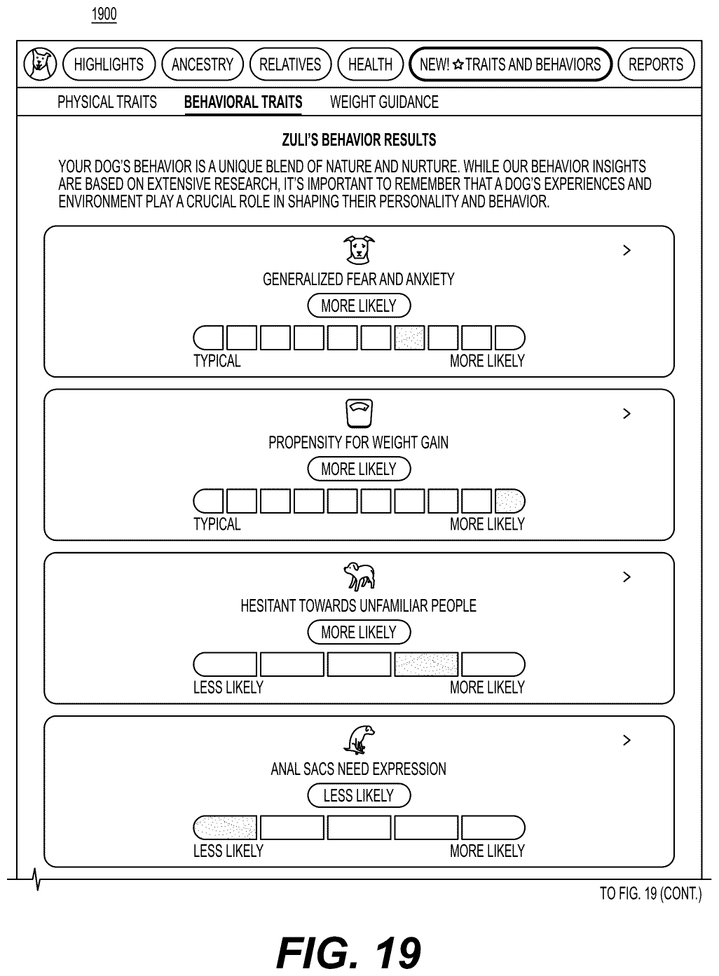

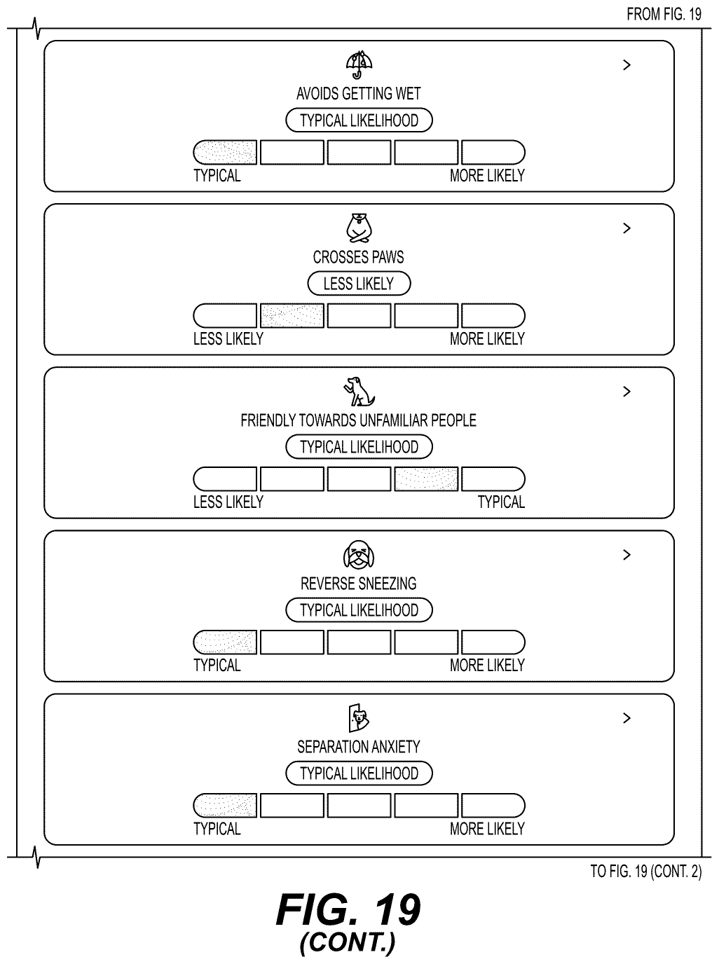

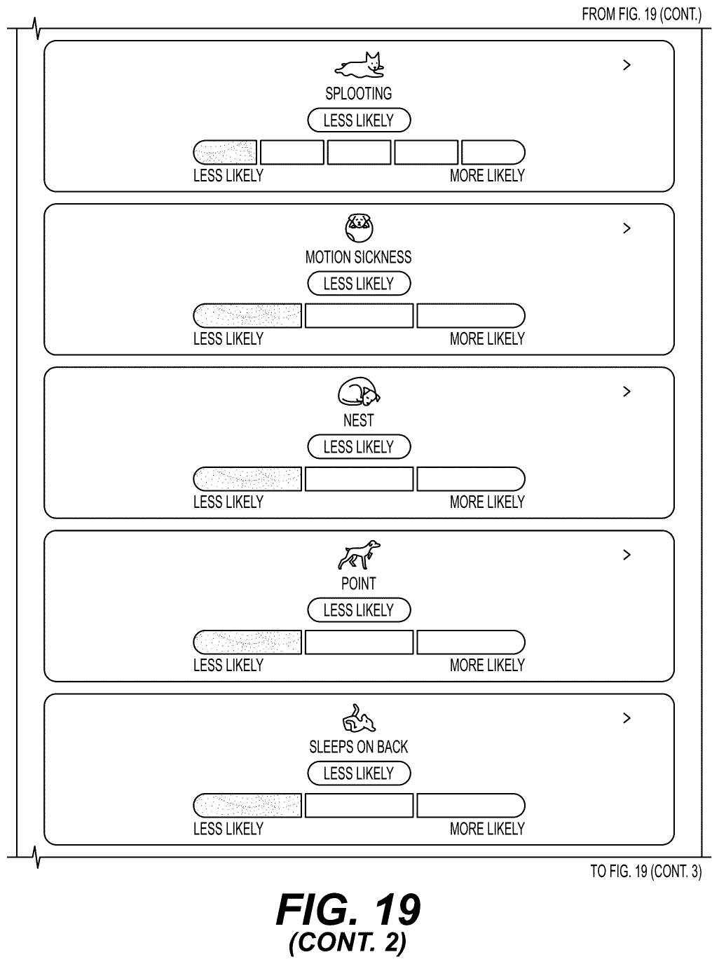

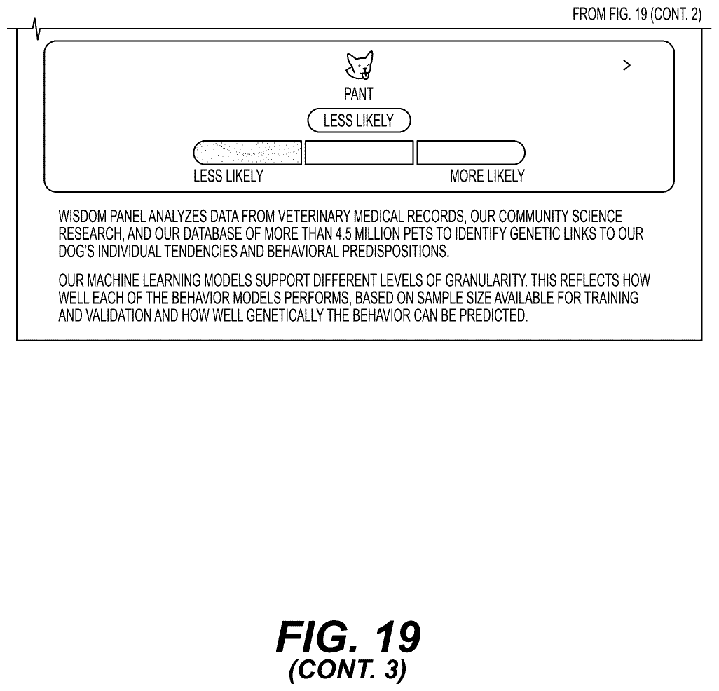

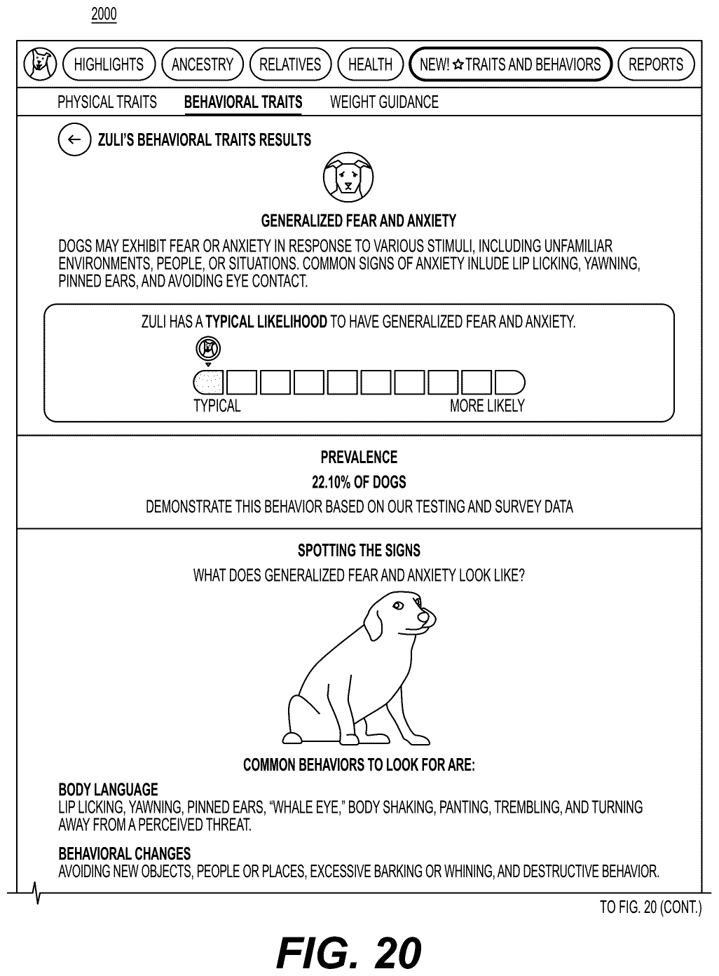

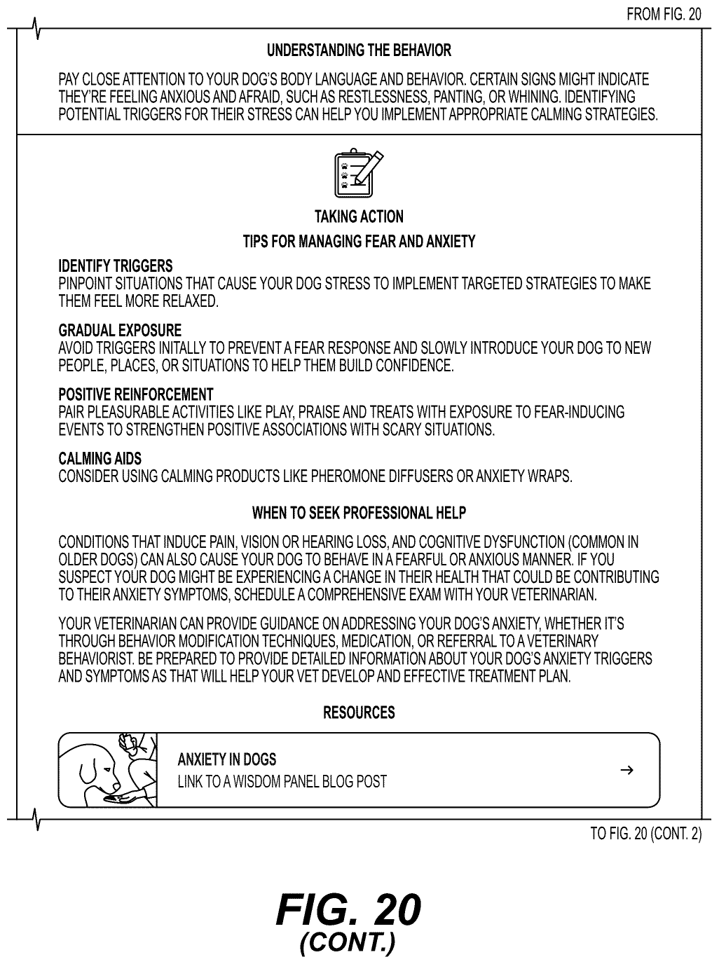

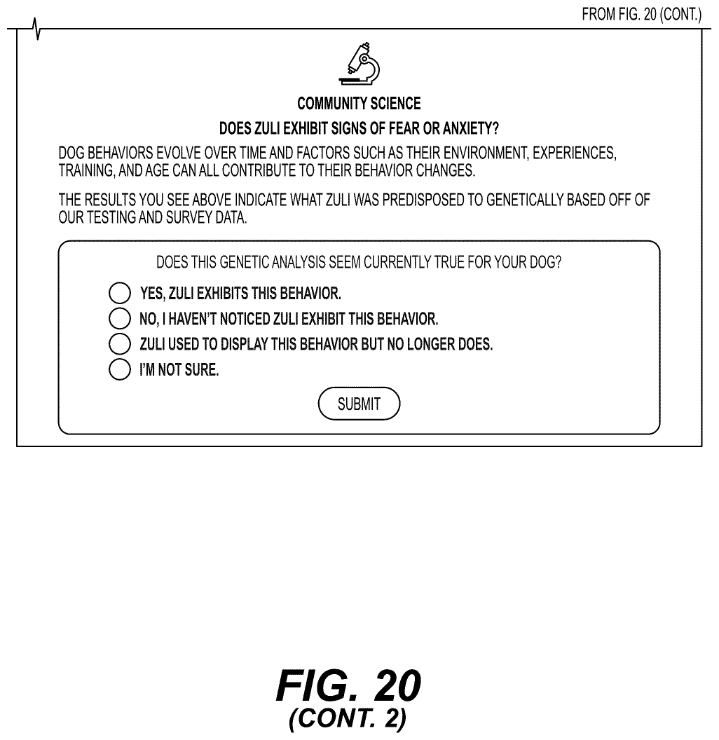

FIG. 19 depicts an exemplary likelihood indication user interface 1900 that may include a listing of the diseases, conditions or other traits that were grouped into the likelihood designation categories. More particularly, each disease, clinical condition or other trait presented on the likelihood indication user interface 1900 may contain an indication of a likelihood designation for developing the disease, clinical condition or other trait that may be represented by a progress bar, where a position of a slider or other similar component along the bar may indicate a level of likelihood of a pet, from a low likelihood to an average likelihood to a high likelihood. Additionally, the progress bar may utilize colors or other similar visual schemes to further visually emphasize the likelihood designation associated with each disease, condition or other trait (e.g., green for low likelihood, yellow for average likelihood, and red for high likelihood). In some embodiments, the bar may indicate a level of likelihood from a typical risk to a more likely likelihood, or a less likely likelihood to a more likely likelihood, with a varying number of levels between. The number of levels in between the outer levels may vary based on the disease, clinical condition or other trait being displayed. The user interface may logically group the diseases, clinical conditions or other traits together (e.g., grouping clinical conditions related to skin, obesity, oral care, mobility, etc.), which may enable a user to quickly obtain information about the likelihoods the pet has for developing a disease, clinical condition or other trait for each classification type. In an aspect, a user may obtain additional information associated with each disease, clinical condition or other trait by, for example, selecting the disease, clinical condition or other trait on the user interface (e.g., via mouse input, touch input, stylus input, etc.). Upon selection, the exemplary condition user interface 2000 may be presented, as shown in FIG. 20. The clinical condition user interface 2000 provides more specific information about the selected disease, clinical condition or other trait, the likelihood of the user's pet developing the selected disease, clinical condition or other trait, and/or how the likelihood for the user's pet compares to other similarly situated pets (e.g., dogs of the same breed, sex, age, etc.) as well as the general population of pets. For example, upon selection of the “Generalized Fear and Anxiety” clinical condition in FIG. 19, the clinical condition user interface 2000 may be presented in FIG. 20 that may include information related to atopic dermatitis, the pet's predicted likelihood of developing Generalized Fear and Anxiety, and/or how the predicted likelihood for the user's pet compares to other similarly situated pets as well as the general population of pets.

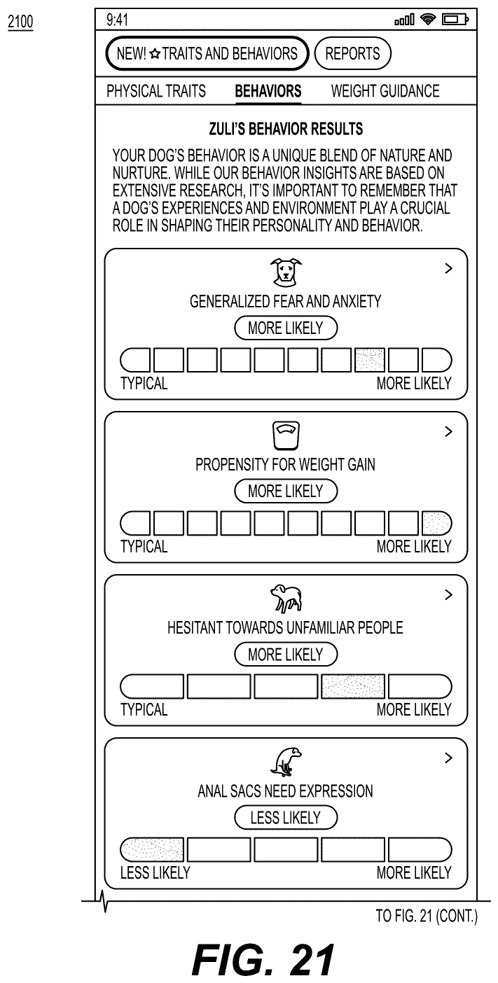

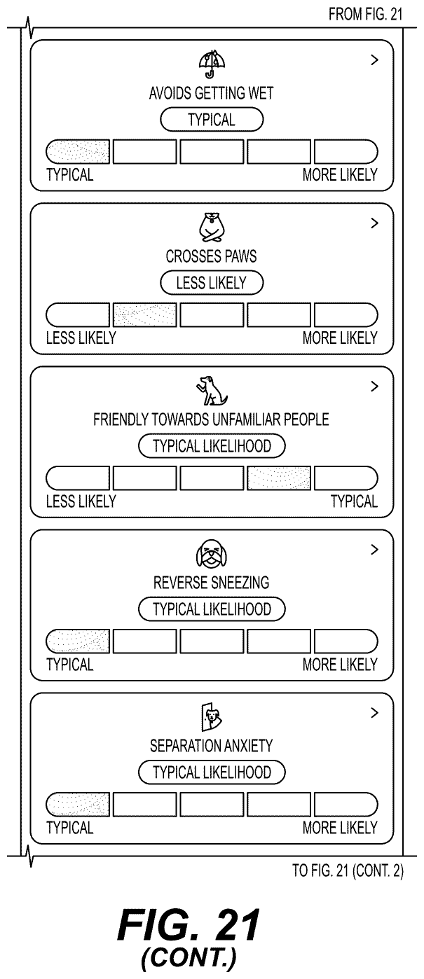

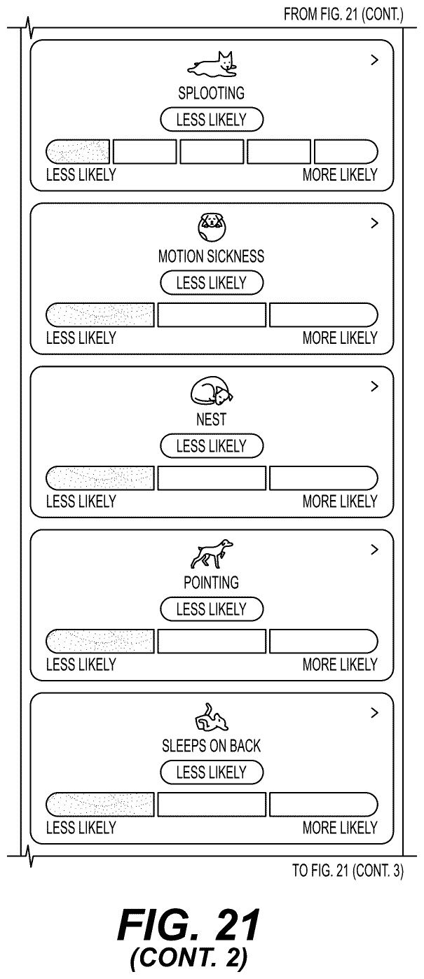

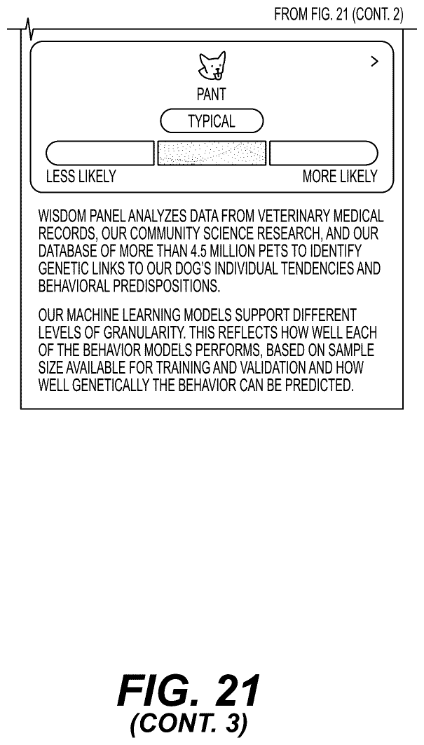

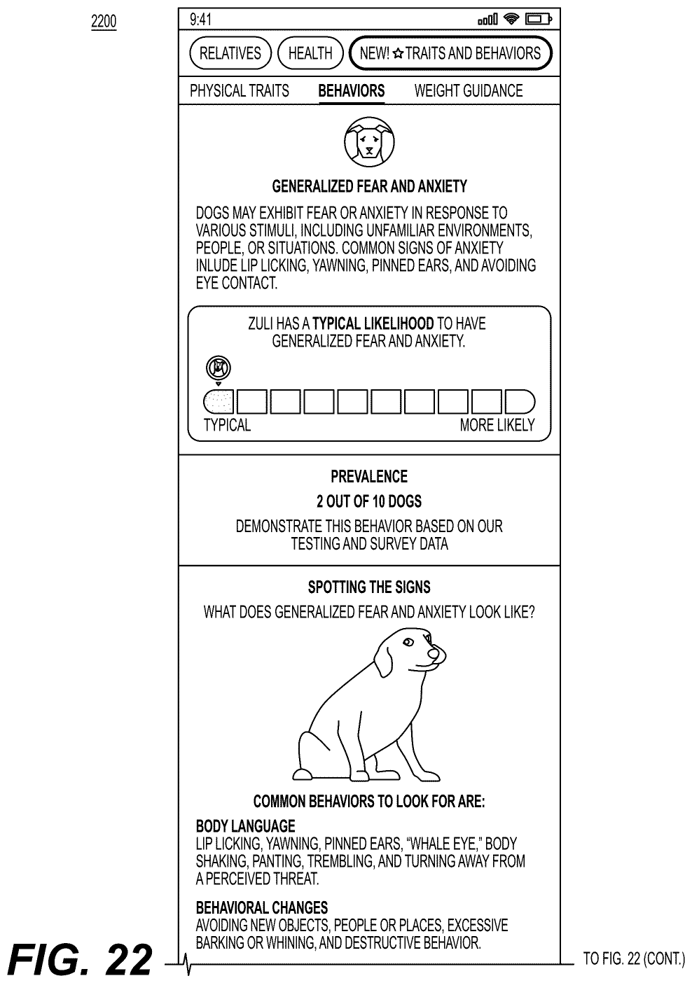

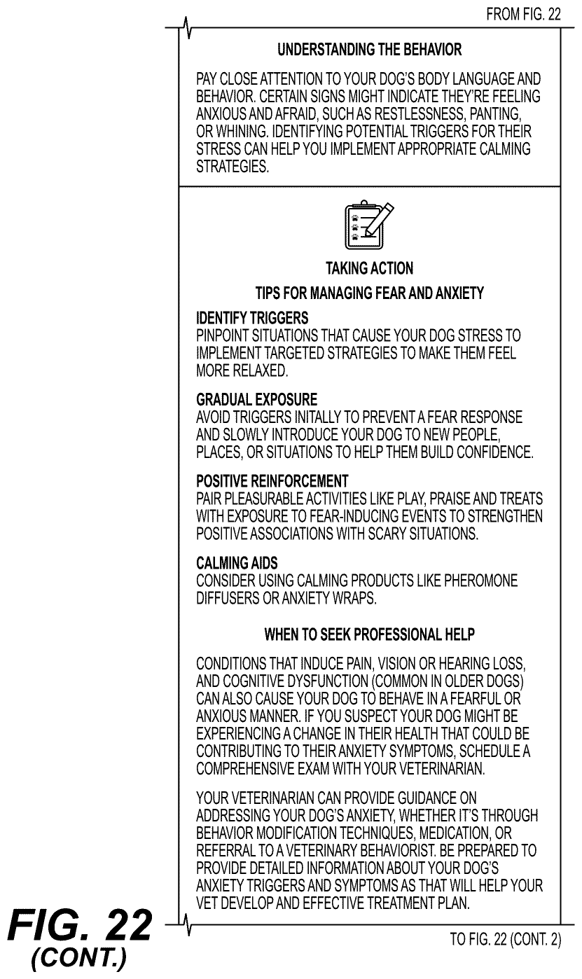

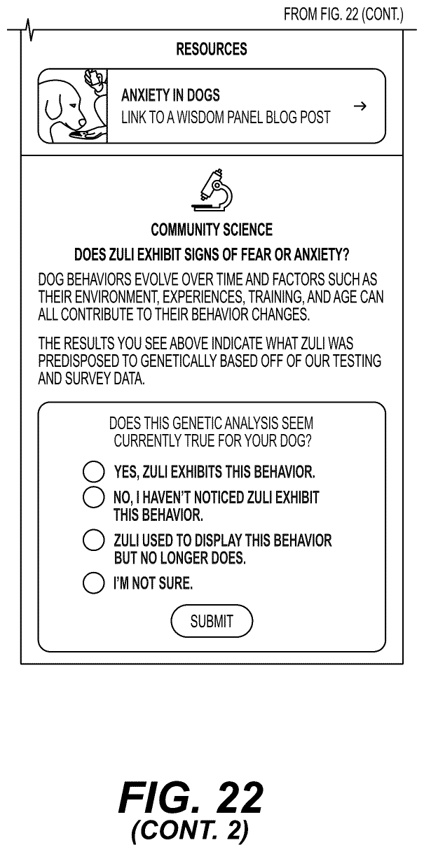

FIG. 21 depicts an exemplary likelihood indication user interface 2100 that may include a listing of the diseases, conditions or other traits that were grouped into the likelihood designation categories. More particularly, each disease, clinical condition or other trait presented on the likelihood indication user interface 2100 may contain an indication of a likelihood designation for developing the disease, clinical condition or other trait that may be represented by a progress bar, where a position of a slider or other similar component along the bar may indicate a level of likelihood from low likelihood to average likelihood to high likelihood. Additionally, the progress bar may utilize colors or other similar visual schemes to further visually emphasize the risk designation associated with each disease, clinical condition or other trait (e.g., green for low likelihood, yellow for average likelihood, and red for high likelihood). In some embodiments, the bar may indicate a level of likelihood from typical to more likely or less likely to more likely, with a varying number of levels between. The number of levels in between the outer levels may vary based on the disease, clinical condition or other trait being displayed. The user interface may logically group the diseases, clinical conditions or other traits together (e.g., grouping clinical conditions related to skin, obesity, oral care, mobility, etc.), which may enable a user to quickly obtain information about the likelihoods the pet has for developing a disease, clinical condition or other trait for each classification type. In an aspect, a user may obtain additional information associated with each disease, clinical condition or other trait by, for example, selecting the disease, clinical condition or other trait on the screen (e.g., via mouse input, touch input, stylus input, etc.). Upon selection, the condition user interface 2200 may be presented, as shown in FIG. 22. The clinical condition user interface 2000 may provide more specific information about the selected disease, clinical condition or other trait, the likelihood of the user's pet developing the selected disease, clinical condition or other trait, and/or how the likelihood for the user's pet compares to other similarly situated pets (e.g., dogs of the same breed, sex, age, etc.) as well as the general population of pets. For example, upon selection of the “Generalized Fear and Anxiety” clinical condition in FIG. 21, the clinical condition user interface 2200 may be presented in FIG. 22 that may include information related to atopic dermatitis, the pet's predicted likelihood of developing Generalized Fear and Anxiety, and how the predicted likelihood for the user's pet compares to other similarly situated pets as well as the general population of pets.

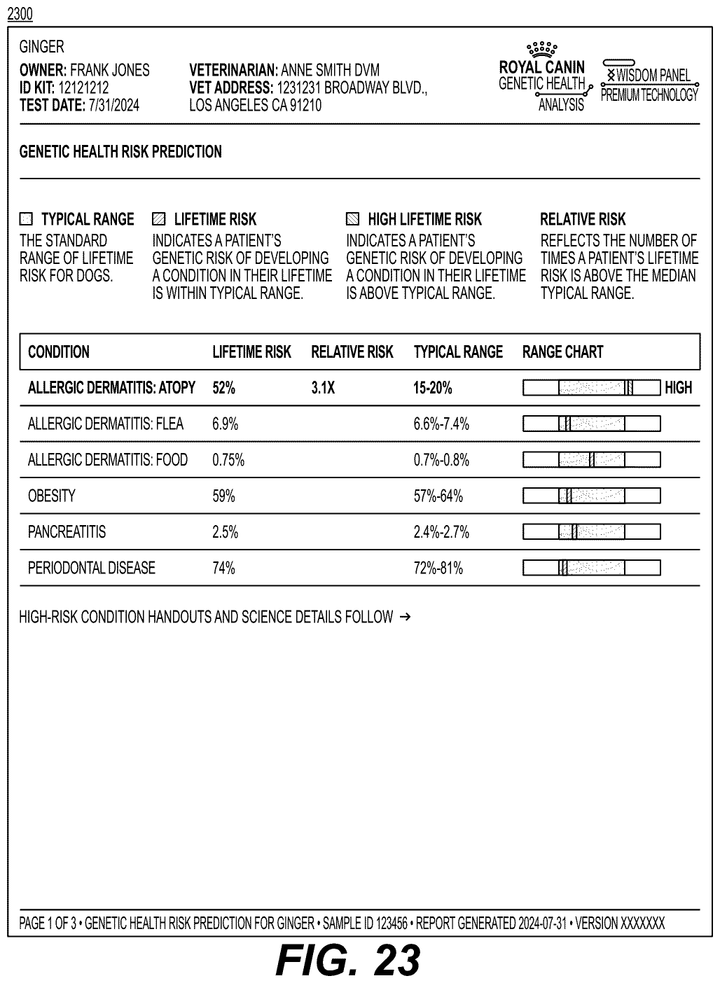

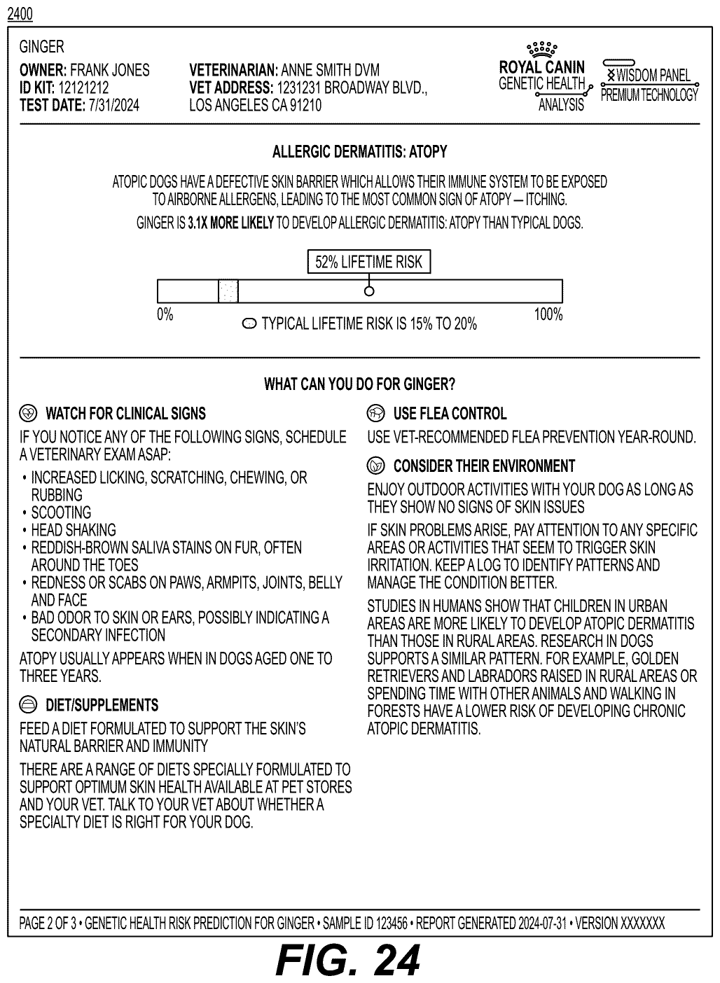

FIG. 23 depicts a genetic health likelihood prediction user interface 2300 that may include a listing of the diseases, conditions or other traits that were grouped into the likelihood designation categories. More particularly, each disease, clinical condition or other trait presented on the likelihood indication user interface 2300 may contain an indication of a likelihood designation for developing the disease, clinical condition or other trait that is represented by a progress bar or a slider bar, where a position of a slider or other similar component along the bar may indicate a level of likelihood. In some embodiments, the level of likelihood may include a range from a low likelihood to an average likelihood to a high likelihood. Additionally, the progress bar may utilize colors or other similar visual schemes to further visually emphasize the risk designation associated with each disease, condition or other trait (e.g., green for low likelihood, yellow for average likelihood, and red for high likelihood). In some embodiments, the bar may indicate a level of likelihood from a typical likelihood to a more likely likelihood, or a less likely likelihood to a more likely likelihood, with a varying number of levels between. The number of levels in between the outer levels may vary based on the disease, clinical condition or other trait being displayed. In an aspect, a user may obtain additional information associated with each disease, clinical condition or other trait by, for example, selecting the disease, clinical condition or other trait on the screen (e.g., via mouse input, touch input, stylus input, etc.). Upon selection, the condition user interface 2400 may be presented, as shown in FIG. 24. The clinical condition screen 2400 may provide more specific information about the selected disease, clinical condition or other trait, the likelihood of the user's pet developing the selected disease, clinical condition or other trait, and how the likelihood for the user's pet compares to other similarly situated pets (e.g., dogs of the same breed, sex, age, etc.) as well as the general population of pets. For example, upon selection of the “Allergic Dermatitis: Atopy” clinical condition in FIG. 23, the clinical condition user interface 2400 may be presented in FIG. 24 that may include information related to Allergic Dermatitis: Atopy, the pet's predicted likelihood of developing atopic dermatitis, and/or how the predicted likelihood for the user's pet compares to other similarly situated pets as well as the general population of pets.

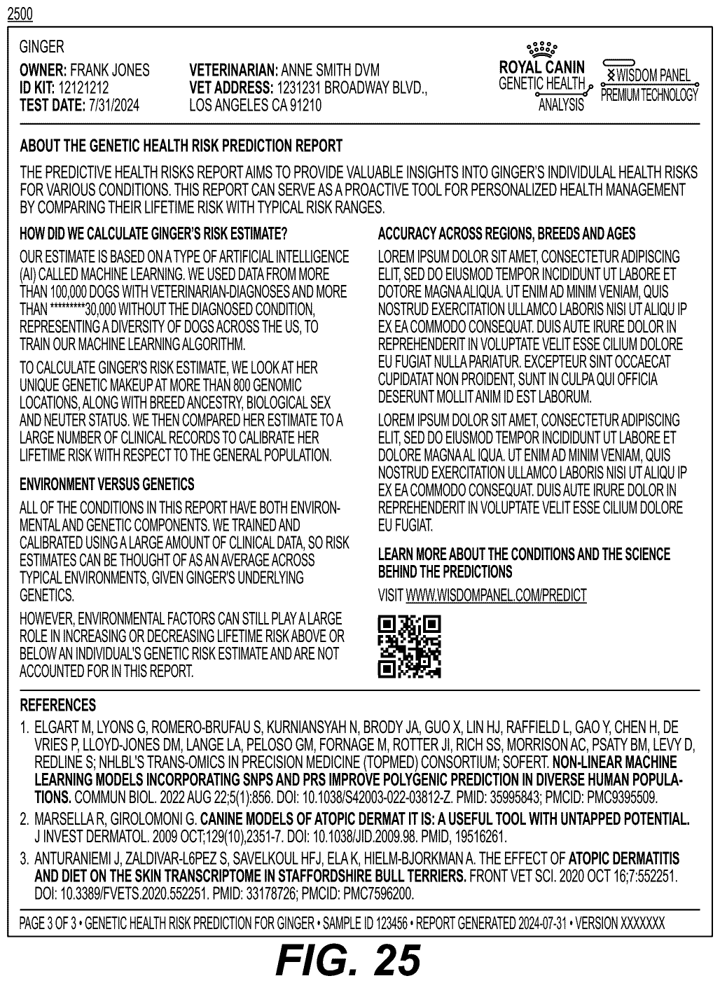

FIG. 25 depicts an exemplary genetic health likelihood prediction user interface 2500 that may include a listing of any relevant information about the genetic health likelihood prediction report. The genetic health likelihood prediction user interface 2500 may include information pertaining to calculating the pet's likelihood estimate, environment versus genetics, accuracy across regions, breeds, and ages, etc. In addition, one or more links and/or interactive images may be presented for user interaction on the genetic health risk prediction user interface 2500.

Calculating the pet's likelihood estimate may include the use of one or more equations, presented below. Model scores may be divided into quantiles (e.g., ranked model scores may be split into bins of equal sample sizes, for example 3, 5, or 10). The likelihood of a condition Lcaseᵢ may be given a population prevalence prev of the condition by calculating the quantile i=1, . . . n as follows, where Ncaseᵢ may include the number of cases in quantile i for a given disease, clinical condition or trait, Ncontrolsᵢ may include the number of controls in quantile i, and Pcasesᵢ and Pcontrolsᵢ may include the estimated population prevalence of cases and controls in quantile i:

$${Pcases}_{i} = ( \frac{N c a s e {s}_{i}}{N c a s e {s}_{i} + N c o n t r o l {s}_{i}} ) * prev \qquad ( 1 )$$

$${Pcontrols}_{i} = ( \frac{N c o n t r o l {s}_{i}}{N c a s e {s}_{i} + N c o n t r o l {s}_{i}} ) * ( 1 - prev ) \qquad ( 2 )$$

$${Lcase}_{i} = ( \frac{P c a s e {s}_{i}}{P c a s e {s}_{i} + P c o n t r o l {s}_{i}} ) \qquad ( 3 )$$

The estimated lifetime likelihood in each bin may then be calculated from the equations above using static estimates of the population prevalence prev for each disease, clinical condition, or trait that may come from either electronic medical record and/or survey data.

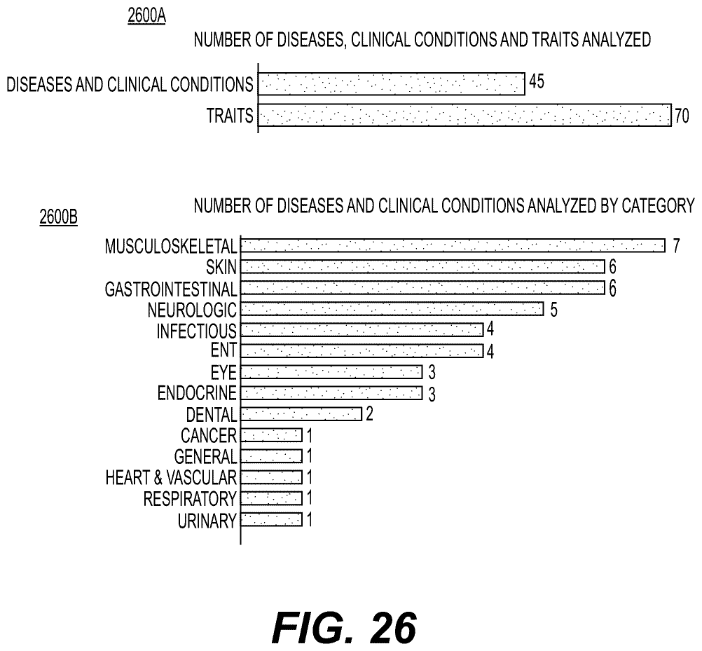

FIG. 26 provides another general overview of the GWAS analysis landscape via a graph 2600A and graph 2600B, which may include an indication of an exemplary number of GWAS analyses conducted across various categories of pet diseases, clinical conditions or other traits. As similarly described above with respect to FIG. 2, these categories are merely representative, and GWAS analyses may be performed for other types and/or categories of pet diseases, clinical conditions or other traits. Additionally, more or fewer GWAS analyses for diseases, clinical conditions or other traits may be performed to enable building of respective machine-learning predictive models for predicting likelihoods of those diseases, clinical conditions or other traits.

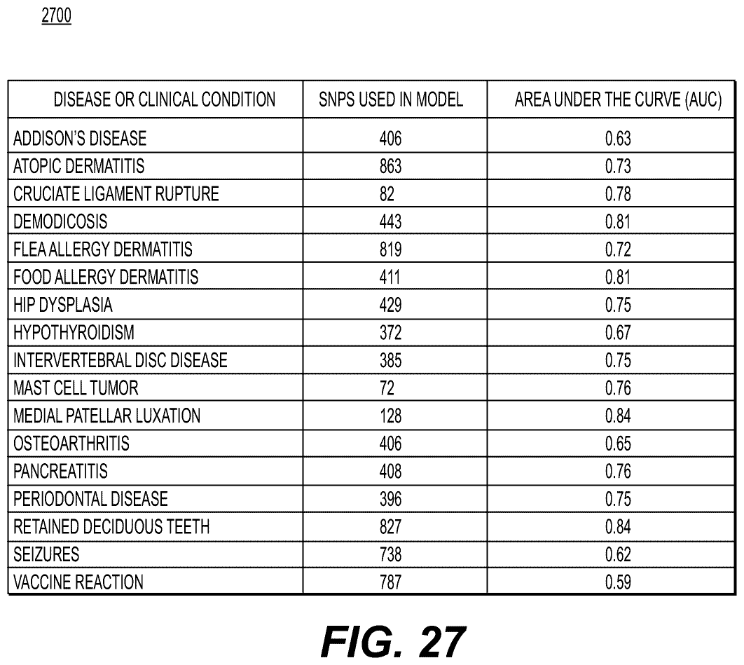

FIG. 27 depicts another illustrative table 2700 providing summary information for each optimal machine-learning predictive model chosen for 17 different diseases, clinical conditions or other traits. As similarly described above with respect to FIG. 8, for each machine-learning predictive model, characteristic information, such as the number of SNPs used in the model, the AUC score, and the number of cases and controls are provided in the table 2700. As briefly alluded to above, the AUC is a metric for classification accuracy of a binary predictive model across all score cutoffs. It measures a curve for all values of apparent true positive rates for equivalent false positive rates. It can vary from 0.5, indicating predictions are effectively random and the model has no predictive value, up to 1, indicating a perfectly predictive classifier. The AUC score provided for reach optimal machine-learning predictive model in the illustrative table 2700 was calculated on a random subset of 10% of input samples held during model training for testing purposes.

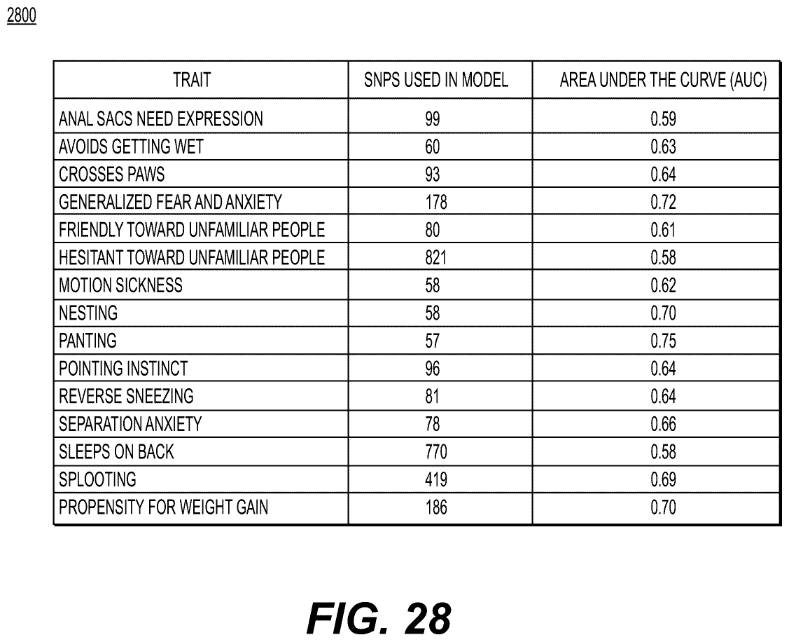

FIG. 28 depicts another illustrative table 2800 providing summary information for each optimal machine-learning predictive model chosen for 15 different diseases, clinical conditions or other traits. As similarly described above with respect to FIG. 8, for each machine-learning predictive model, characteristic information such as the number of SNPs used in the model, the AUC score, and the number of cases and controls are provided. As briefly alluded to above, the AUC is a metric for classification accuracy of a binary predictive model across all score cutoffs. It measures a curve for all values of apparent true positive rates for equivalent false positive rates. It can vary from 0.5, indicating predictions are effectively random and the model has no predictive value, up to 1, indicating a perfectly predictive classifier. The AUC score provided for reach optimal machine-learning predictive model in the illustrative table 2800 was calculated on a random subset of 10% of input samples held during model training for testing purposes.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some aspects described herein include some but not other features included in other aspects, combinations of features of different aspects are meant to be within the scope of the invention, and form different aspects, as would be understood by those skilled in the art. For example, in the following claims, any of the claimed aspects can be used in any combination.

Thus, while certain aspects have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for identifying a likelihood of a pet developing at least one of a disease, a clinical condition or other trait, comprising:
receiving, at a server system, pet data associated with the pet, wherein the pet data includes genetic data and breed data;
generating, using a processor of the server system, a likelihood score for the pet associated with the at least one of the disease, the clinical condition or other trait by applying the pet data to a trained machine learning model, wherein the trained machine learning model is trained to predict likelihood scores for the at least one of the disease, the clinical condition or other trait; and
causing, by the processor of the server system, a visual representation of the likelihood score to be displayed on a user device, wherein the visual representation includes an indication of the likelihood of the pet developing the at least one of the disease, the clinical condition or other trait.

2. The computer-implemented method of claim 1, wherein the pet data further comprises one or more other data types including: clinical history data, demographic information, medical history information, or lifestyle factor information.

3. The computer-implemented method of claim 1, wherein the receiving the pet data further comprises:
generating, using the processor, at least one guided question related to pet health;
causing the user device to display the at least one guided question; and
receiving, in response to the at least one guided question, an answer from the user device.

4. The computer-implemented method of claim 1, wherein the trained machine learning model is trained based on at least a portion of a plurality of training datasets associated with a plurality of pets, each of the plurality of training datasets including one or more data types associated with a respective pet, including: genetic data comprising single nucleotide polymorphism (SNP) data for the respective pet determined to be relevant based on a genome-wide association study (GWAS) for the at least one of the disease, the clinical condition or other trait, breed data for the respective pet, clinical history data for the respective pet, demographic information for the respective pet, medical history information for the respective pet, or lifestyle factor information for the respective pet.

5. The computer-implemented method of claim 1, wherein the trained machine learning model is of a type selected from the group consisting of: a gradient boosting model, a random forest model, a neural network, a logistic regression model, a support vector machine, a decision tree model, and an Extreme Gradient Boosting model.

6. The computer-implemented method of claim 1, wherein the visual representation of the likelihood score comprises at least one of: a text-based likelihood indicator, a color-coded likelihood indicator, or a progress bar indicator.

7. The computer-implemented method of claim 1, further comprising extracting, from the genetic data, a subset of single nucleotide polymorphism (SNP) data determined to be relevant based on a genome-wide association study (GWAS) for the at least one of the disease, the clinical condition or other trait, wherein the subset of SNP data and the breed data is applied to the trained machine learning model.

8. The computer-implemented method of claim 1, further comprising generating one or more recommendations based on the likelihood score and causing the one or more recommendations to be displayed on the user device.

9. The computer-implemented method of claim 8, wherein the one or more recommendations include one or more suggested actions for improving health of the pet.

10. The computer-implemented method of claim 1, further comprising:
receiving, at the server system, new pet data associated with the pet; and
updating, based on the received new pet data, the likelihood score.

11. A computer system for identifying a likelihood of a pet developing at least one of a disease, a clinical condition or other trait, the computer system comprising:
at least one processor; and
at least one memory storing instructions that are executable by the at least one processor, cause the at least one processor to:
receive pet data associated with the pet, wherein the pet data includes genetic data and breed data;
generate, by applying the pet data to a trained machine learning model, a likelihood score for the pet associated with the clinical condition, wherein the trained machine learning model is trained to predict likelihood scores for the at least one of the disease, the clinical condition or other trait; and
cause a visual representation of the likelihood score to be displayed on a user device, wherein the visual representation includes an indication of the likelihood of the pet developing the at least one of the disease, the clinical condition or other trait.

12. The computer system of claim 11, wherein the pet data further comprises one or more other data types including: clinical history data, demographic information, medical history information, and lifestyle factor information.

13. The computer system of claim 11, wherein to receive the pet data, the at least one processor is further caused to:
generate at least one guided question related to pet health;
cause the user device to display at least one guided question; and
receive, in response to the at least one guided question, an answer from the user.

14. The computer system of claim 11, wherein the trained machine learning model is trained based on at least a portion of a plurality of training datasets associated with a plurality of pets, each of the plurality of training datasets including one or more data types associated with a respective pet, including: genetic data comprising single nucleotide polymorphism (SNP) data for the respective pet determined to be relevant based on a genome-wide association study (GWAS) for the clinical condition, breed data for the respective pet, clinical history data for the respective pet, demographic information for the respective pet, medical history information for the respective pet, or lifestyle factor information for the respective pet.

15. The computer system of claim 11, wherein the trained machine learning model is of a type selected from the group consisting of: a gradient boosting model, an extreme gradient boosting model, a random forest model, a neural network, a logistic regression model, a support vector machines, a decision tree model, and an Extreme Gradient Boosting model.

16. The computer system of claim 11, wherein the visual representation of the likelihood score comprises one or more of: a text-based likelihood indicator, a color-coded risk indicator, a progress bar indicator.

17. The computer system of claim 11, wherein the instructions are further executable by at least the processor to cause the at least one processor to:
generate one or more recommendations based on the likelihood score; and
cause the one or more recommends to be displayed on the user device.

18. The computer system of claim 17, wherein the one or more recommendations include one or more suggested actions for improving health of the pet.

19. The computer system of claim 11, wherein the instructions are further executable by the at least one processor to cause the at least one processor to:
receive new pet data associated with the pet; and
update, based on the received new pet data, the likelihood score.

20. A non-transitory computer-readable medium storing computer-executable instructions which, when executed by at least one processor, cause the at least one processor to perform operations comprising:
receiving pet data associated with the pet, wherein the pet data includes genetic data and breed data;
generating a likelihood score for the pet associated with at least one of a disease, a clinical condition or other trait by applying the pet data to a trained machine learning model, wherein the trained machine learning model is trained to predict likelihood scores for the at least one of the disease, the clinical condition or other trait; and
causing a visual representation of the likelihood score to be displayed on a user device, wherein the visual representation includes an indication of a likelihood of the pet developing the at least one of the disease, the clinical condition or other trait.
